(19) United States
(12) Patent Application Publication
(10) Pub. No.: US 2026/0335414 A1
(43) Pub. Date: Oct. 29, 2026

(54) METHODS OF TREATING INFLAMMATORY EYE DISEASES

(71) Applicants: MOR RESEARCH APPLICATIONS LTD., Ramat Gan (IL); THE SCRIPPS RESEARCH INSTITUTE, La Jolla, CA (US)

(72) Inventors: Tami LIVNAT, Savyon (IL); Dov WEINBERGER, Tel Aviv (IL); Yael NISGAV, Ramat Hasharon (IL); John H. GRIFFIN, Del Mar, CA (US)

(21) Appl. No.: 19/109,083

(22) PCT Filed: Sep. 6, 2023
(86) PCT No.: PCT/IB2023/058826

§ 371 Date: Mar. 5, 2025

Related U.S. Application Data
(60) Provisional application No. 63/374,651, filed on Sep. 6, 2022.
(60) Provisional application No. 63/504,288, filed on May 25, 2023.

(51) Int. Cl. A61K 38/36; A61K 39/395; A61K 45/06; A61P 25/28; A61P 27/02

(52) U.S. Cl. CPC A61K 38/36; A61K 39/3955; A61K 45/06; A61P 25/28; A61P 27/02

(57) ABSTRACT

Methods for treating inflammation and conferring neuroprotection in the eye are provided, comprising the systemic and/or local administration of a variant of activated protein C (APC). Certain diseases, disorders or conditions caused directly by inflammation and/or neurodegeneration or featuring development of inflammation and/or neurodegeneration as a secondary stage or a complication thereof may be treated by a disclosed method, for example, retinal and/or choroidal diseases and disorders such as retinopathy, age-related macular degeneration (AMD), neurodegenerative disorders of the retina and optic nerve injury.

### FIELD OF THE INVENTION

The present disclosure relates to the use of a variant of activated protein C (APC) in treatment of ocular diseases, particularly, but not exclusively in treatment of inflammatory eye diseases.

### BACKGROUND

Activated Protein C (APC) is a physiological anticoagulant derived from its zymogen protein C (PC). Anticoagulant, cytoprotection and cell signaling are major APC activities, of which anticoagulation and cell signaling are well defined. Inhibition of inflammatory responses, endothelial barrier protection, and increased cell survival are key mechanisms underlying the cellular protective effects of APC.

Congenital PC deficiency infants were diagnosed with various ocular pathologies of the anterior and posterior segments, pointing to the importance of endogenous PC during or after fetal development of the eye. Children born with severe PC deficiency are very often blind.

As APC is a natural coagulation inhibitor, bleeding risk may limit its use as a therapeutic agent. 3K3A-APC is a recombinant engineered variant of APC (three Lys residues replace three Ala residues) with markedly reduced anticoagulant activity. The replacement of the three residues in the 3K3A-APC variant reduces APC's interactions with the clotting factor Va and diminishes its anticoagulant activity. Importantly, 3K3A-APC sustains PC's pleiotropic cytoprotective activities and preserves the interactions with its cell receptors, including binding to endothelial protein C receptor (EPCR) and activating protease-activated receptors (PARs) PAR-1 and PAR-3 (Mosnier et al., Blood, 104(6): 1740-1744, 2004; Griffin et al., Blood, 125:2898-2907, 2015 and Blood, 132:159-169,2018).

Beneficial cytoprotective activities of 3K3A-APC that manifest as anti-inflammatory, anti-apoptotic, endothelial and epithelial cell barrier protection, and its regenerative effects are reported in many disease models of different organs. Furthermore, the multiple neuroprotective actions of 3K3A-APC led to successful translation from preclinical to phase II clinical studies for acute ischemic stroke.

For example, the anti-inflammatory effects of APC and 3K3A-APC have long been appreciated in non-ocular tissues. APC's broad-spectrum activities include inhibition of nuclear factor kappa B (NF-κB) activation, changes in gene expression profiles, downregulation of adhesion molecules on endothelial cells, reduced leukocyte adhesion and infiltration, inhibition of neutrophil NETosis, and cleavage of extracellular histones. Recent studies suggest that APC and 3K3A-APC can express anti-inflammatory effects via suppressing the NLRP3 inflammasome.

U.S. Patent Publication No. 2021/0206834 of the present inventors discloses the effects of 3K3A-APC in the retina and demonstrates that 3K3A-APC inhibits and regresses choroidal neovascularization (CNV) growth and preserves wild-type (wt) plasma-derived APC's protective activities in the retina in a murine model of laser-induced CNV. Furthermore, the present inventors have previously reported that 3K3A-APC and wt-APC significantly reduced vascular endothelial growth factor (VEGF) levels at CNV sites (Livnat et al., Biomolecules, 11(3), 2021; Livnat et al., Exp Eye Res, 186:107695, 2019).

Currently, no data is available regarding the anti-inflammatory effects of APC or a mutant thereof in ocular pathologies. As accumulating evidence implicates that inflammatory activation contributes to tissue damage in many ocular diseases, including uveitis, glaucoma, diabetic retinopathy and age-related macular degeneration (AMD), there is a growing need to target ocular inflammation using novel treatment strategies.

### SUMMARY

3K3A-Activated Protein C (APC) is a recombinant variant of the physiological anticoagulant APC with pleiotropic cytoprotective properties, yet without bleeding risks. The present inventors have shown, for the first time, that the variant 3K3A-APC reduces undesired or deleterious innate immune response in the eye and confer cytoprotection and, thereby, hold the potential for treating myriad of ophthalmic pathologic situations, directly or indirectly associated with excessive inflammatory response and/or leukocyte infiltration to the eye. Since inflammation is not only relevant in autoimmune conditions such as uveitis but is recognized as a major driving force behind retinal degenerative diseases such as diabetic retinopathy and age-related macular degeneration, these findings have clinical implications for the treatment of retinal pathologies associated with inflammation.

In an aspect, the present disclose relates to methods for treating various pathologies of the eye caused by, or otherwise associated with, inflammation. Specifically, embodiments described herein pertain to the use of an APC variant, or a functional partial sequence thereof, in treatment of ocular inflammation, for example, ocular inflammation associated with an ocular or non-ocular disease disorder or condition. Exemplary diseases, disorder and conditions include, but are not limited to, anterior, intermediate and posterior uveitis, panuveitis, endogenous and exogenous endophthalmitis, inflammatory diseases of the optic nerve such as optic neuritis, papilledema, anterior and ischemic optic neuropathy (AION), choroidal neovascularization (CNV), wet age-related macular degeneration (nAMD), dry AMD, pathologic myopia, pseudoxanthoma elasticum with angioid streaks, Beh et's disease, retinitis pigmentosa, glaucoma, best Vitelliform macular degeneration (BVMD), Stargard's disease, choroiditis, episcleritis, scleritis, thyroid ophthalmopathy and retinopathy.

Further contemplated herein is the use of an APC variant or a functional partial sequence thereof in methods for treating various ocular diseases, disorders or conditions such as, but not limited to, macular edema associated diseases, ischemic retinopathy, retinal leakage, blood vessel occlusion, oxidative damage, damage to the optic nerve and the death of associated retinal ganglion cells (RGCs) by elevated intraocular pressure (IOP), chronic inflammation, an autoimmune condition such as sarcoidosis or systemic lupus erythematosus (SLE), temporal arteritis (e.g., giant cell arteritis (GCA)), Lyme disease, viral infection, cat scratch fever (Bartonella), syphilis, bacterial infection, Herpes virus, exogenous or endogenous endophthalmitis, an accidental, occasional incidence in which inflammation develops following a traumatic injury of the retina, perforating and blunt trauma, complications during or post ophthalmic medical procedure, or drugs side effects. At least some of these diseases, disorders and conditions are associated with inflammatory processes in the eye.

The present disclosure further relates to methods for treatment of an ocular pathology associated with activation of retinal NLRP3 inflammasome and/or translocation and activation of retinal microglia cells, whereby to a subject in need thereof is provided with a therapeutically effective amount of a variant of APC or a functional partial sequence thereof. The ocular pathology thus treated may be, for example, an ocular disease, disorder and/or condition associated with activation of retinal NLRP3 inflammasome and/or translocation and activation of retinal microglia cells such as, but not limited to, AMD and CNV.

For example, CNV may be treated by inhibiting translocation and activation of retinal microglia cells and thereby regression of choroidal neovascularization growth and CNV-associated leakage is inhibited or prevented.

In a further aspect, the present disclosure relates to a method for cytoprotection, comprising administering a therapeutically effective amount of a variant of APC, or a functional partial sequence thereof, to a subject in need thereof. In some embodiments, cytoprotection is conferred to neuronal cells, for example, neuronal cells of the eye. In accordance with these embodiments, a disclosed method provides neuroprotection to neurons and neurovascular units (NVUs) of the eye and is useful in treating neurodegenerative disorders of the retina, optic nerve injury (for example, mechanical injury), death of retinal ganglion cells (RGCs), damages caused by exposure to toxins and impairments due to genetic mutations.

In any of the embodiments described herein, the APC variant or a functional partial sequence thereof used in a contemplated method is administered systemically, for example by intravenous injection.

In some embodiments, the APC variant is 3K3A-APC.

### BRIEF DESCRIPTION OF THE DRAWINGS

Some embodiments are herein described, by way of example only, with reference to the accompanying drawings. With specific reference now to the drawings in detail, it is stressed that the particulars shown are byway of example and for purposes of illustrative discussion of embodiments. In this regard, the description taken with the drawings makes apparent to those skilled in the art how embodiments may be practiced.

In the drawings:

FIG. 1 are representative indirect ophthalmoscopy fundus images of a naïve mouse eye (control), and of an eye 24 hours post-endotoxin (LPS) induced uveitis (EIU);

FIG. 2 are exemplary images of retinal flatmounts of mice eyes taken 24 hours post-EIU, with or without 3K3A-APC intravitreal (ITV) injection applied 1 hour prior LPS injection. Retinal blood vessels were stained using fluorescein isothiocyanate (FITC)-dextran perfusion (green), and leukocytes were stained using anti-CD11b antibody (red). Left panel demonstrates upper view (scale bar=50 μm) and right panel represents Z-plane (depth) (scale bar=70 μm);

FIGS. 3A-3E are bar graphs (3A-3C, 3E) and images (3D) showing the effect of 3K3A-APC on leukocyte number and extravasation in endotoxin-induced uveitis (EIU). The total number and localization of CD11b positive leukocytes was assessed naïve eyes (Control), eyes damaged by EIU but not treated with 3K3A-APC (LPS) and eyes subjected to EIU and treated with 3K3A-APC (LPS+3K3A-APC). 3A, 3B, 3C and 3E: quantitative analysis of CD11b⁺ cell counts within the retinal parenchyma, co-localization of CD11b⁺ leukocytes to blood vessels, retinal thickness and CD11b⁺ cell counts within the retinal parenchyma when 3K3A-APC was administered 4 h EIU injection, respectively. Data are presented as mean±SD (n=5-9 per group) and were analyzed using one-way ANOVA followed by Tukey post hoc test. 3D: representative upper view images of retinal flatmounts eyes taken 24 hours post-EIU, with or without 3K3A-APC intravitreal (ITV) treatment applied 4 hours post-EIU. Retinal blood vessels were stained using fluorescein isothiocyanate (FITC)-dextran perfusion (green), and leukocytes were stained using anti-CD11b antibody (red);

FIGS. 4A-4B are dot plots (4A) and bar graphs (4B) showing cell subpopulations in mice eyes as obtained by flow-cytometry analysis. LPS: mice in which uveitis was induced by ITV injection of lipopolysaccharide (LPS); LPS+3K3A-APC: mice treated ITV with the mutant 3K3A prior to LPS injection; Control: mice which did not receive any treatment. Single cell preparations were analyzed by flow-cytometry 24 hours after ITV injections of LPS. In 4A, the two vertical dot plots on the left, indicate the gating strategy to exclude debris and dead cells using 7AAD negative staining. 4B: statistical analysis of total CD45⁺ leukocytes. Data are presented as mean±SD and analyzed using mixed-effects models followed by Tukey post-hoc test (n=6-7 pooled retinas per group, with a total of 5 experiments repeated);

FIGS. 5A-5C are images (5A) and bar graphs (5B, 5C) showing microglia cell accumulation and activation in retinal cryosection. 5A: retinal cryosection images taken 24-hours after intravitreal injection (ITV) of LPS to eyes of mice, either preceded by ITV injection of 3K3A-APC (LPS+3K3A-APC) or not (LPS). Control: mice which did not receive any treatment. Microglial cells were stained for a specific marker, Iba1. 5B: statistical analysis—microglial residence area calculated using values of 4 microscopic fields (2 fields×2 slides). The data are presented as mean±SD, analysis was done using one-way ANOVA followed by Tukey post hoc test (n=4-5 per group). 5C: statistical analysis—the number of Iba1 positive cells (i.e., microglia), both active and quiescent, in outer retina. The data are presented as mean±SD and analyzed using unpaired two-tailed Student's t-test (n=4-5 per group). GCL—ganglion cell layer; INL—inner nuclear layer; IPL—inner plexiform layer; ONL—outer nuclear layer; OPL—outer plexiform layer;

FIGS. 6A-6C are images (6A, 6B) and bar graphs (6C, 6D) showing NLRP3 inflammasome and IL1-β levels in mice retina following EIU. Retinal cryosections were stained using direct antibodies against NLRP3 (6A) or IL1-β (6B). Retinal cryosection images taken 24-hours after ITV injection of LPS to eyes of mice, either preceded by ITV injection of 3K3A-APC (LPS+3K3A-APC) or not (LPS). Control: mice which did not receive any treatment. 6C, 6D: statistical analysis—NLRP3 and IL1-β area were calculated using values of 4 microscopic fields (2 fields×2 slides) which were averaged. Data are presented as mean±SD and analyzed using one-way ANOVA followed by Tukey post hoc test (n=4-6 per group);

FIG. 7 are images (upper view) of exemplary retinal flatmount of mice eyes taken 24 hours post-EIU induction followed by intravenous administration via tail vein of either 3K3A-APC or vehicle (LPS+3K3A-APC and LPS, respectively), 10 min and 4 hours after EIU induction. The left image (Control) is of eye which did not receive any treatment; the middle image is of eye subjected to EIU induction without 3K3A-APC pretreatment (LPS); and the right image is of eye subjected to EIU induction and treated with 3K3A-APC (LPS+3K3A-APC). Retinal blood vessels were stained green using FITC-dextran perfusion, and inflammatory cells were stained red using anti-CD11b antibody (scale bar=50 μm);

FIGS. 8A-8C are images (8A) and bar graphs (8B, 8C) showing the effect of intravenously administered 3K3A-APC on laser-induced choroidal neovascularization (CNV). 8A: representative color immunofluorescent images of retinal pigment epithelium (RPE)-choroid flatmounts upper view taken from control (left image (Control)) eyes and eyes subjected to laser with or without 3K3A-APC treatment (middle (Laser) and right (laser+3K3A-APC) images, respectively). CNV was stained green using FITC-dextran, and inflammatory cells were stained red using anti-CD11b antibody. 8B, 8C: quantifications of CNV depth (μm) and CD11b positive cell number, respectively. Data are presented as mean±SD (4-5 mice per group). Data were analyzed using one-way ANOVA followed by Tukey post hoc test;

FIGS. 9A-9B are images (9A) and a box plot (9B) showing the effect of intravitreally administered 3K3A-APC on NLRP3 levels in a laser-induced CNV lesion in murine eyes. 9A: representative images of retinal cryosections immunostained with NLRP3 antibodies. Blue represents cell nuclei; green represents NLRP3 (scale bar=100 μm). In the laser treated eyes, NLRP3 staining at the CNV site is marked with an asterisk. The left image in the upper panel (Control) is of an untreated eye (i.e., without any intervention); the middle and right images (Laser) are of CNV-induced eye not treated with 3K3A-APC showing the CNV site and CNV margins, respectively; and the lower two images are of CNV-induced eye (CNV site and CNV margins, respectively) treated with 3K3A-APC (Laser+3K3A-APC). 9B: quantitative analysis of the NLRP3 area (μm²). Positive staining areas were calculated using values from 4 microscopic fields (2 fields/slides×2 slides/animal), which were averaged and used as raw data for further analysis. Results are presented as box-and-whiskers plots. The boxes span the 25th to the 75th percentile (or Quartile 1 to Quartile 3), the line inside each box denotes the median, and the whiskers span the lowest to the highest observations. Comparisons were performed with the Kruskal-Wallis test followed by Dunn's post hoc test (n=5-6 per group). GCL—ganglion cell layer; INL—inner nuclear layer; IPL—inner plexiform layer; NFL—nerve fiber layer; ONL— outer nuclear layer; OPL—outer plexiform layer; RPE—retinal pigment epithelium;

FIGS. 10A-10B are images (10A) and a box plot (10B) showing the effect of intravitreally administered 3K3A-APC on IL-1β levels in a laser-induced CNV lesion in murine eyes. 10A: representative images of retinal cryosections immunostained with IL-1β antibodies. Blue represents cell nuclei; green represents IL-1β (scale bar=100 μm). In the laser treated eyes, IL-1β staining at the CNV site is marked with an asterisk. The left image (Control) is of an untreated eye (i.e., without any intervention); the two middle images (Laser) are of CNV-induced eye not treated with 3K3A-APC (the right image is the zoomed-in section marked on the left image); and the right image is of CNV-induced eye treated with 3K3A-APC (Laser+3K3A-APC). 10B: quantitative analysis of IL-1β area (μm²). Positive staining areas were calculated using values from 4 microscopic fields (2 fields/slides×2 slides/animal), which were averaged and used as raw data for further analysis. Results are presented as box-and-whiskers plots. The boxes span the 25th to the 75th percentile, the line inside each box denotes the median, and the whiskers span the lowest to the highest observations. Comparisons were performed with the Kruskal-Wallis test followed by Dunn's post hoc test (n=5-6 per group). GCL—ganglion cell layer; INL—inner nuclear layer; IPL—inner plexiform layer; NFL—nerve fiber layer; ONL—outer nuclear layer; OPL—outer plexiform layer; RPE—retinal pigment epithelium;

FIGS. 11A-11C are images (11A, 11B) and a box plot (11C) showing the effect of intravitreally administered 3K3A-APC on microglia recruitment and activation at a CNV lesion. 11A: representative images of retinal cryosections immunostained with Iba1 antibodies, a specific marker for microglia (green), and DAPI (blue) as a nuclei marker. The right image of each group represents a higher magnification (scale bar=50 μm) of the left image (scale bar=100 μm). The left couple of images (Control) are of an untreated eye (i.e., without any intervention); the middle couple of images (Laser) are of CNV-induced eye untreated with 3K3A-APC; and the right couple of images (Laser+3K3A-APC) are of CNV-induced eye treated with 3K3A-APC. The CNV site is marked with an asterisk, and amoeboid, active microglial cells are marked by an arrow. 11B: upper views (upper panel of images) and depth Z-view (lower panel of images) of representative RPE-choroid flatmount immunostained with Iba1 antibodies (red) and scanned from the RPE into the choroid. Intravitreal injections of either 3K3A-APC or saline were administered one hour after CNV induction. The left two stacked images (Control) are of an untreated eye (i.e., without any intervention); the 2 stacked images in the middle (Laser) are of CNV-induced eye untreated with 3K3A-APC; and the 2 stacked images on the right (Laser+3K3A-APC) are of CNV-induced eye treated with 3K3A-APC. 11C: quantitative analysis of Iba1⁺ cells area (μm²) presented as box-and-whiskers plots. The line inside each box denotes the median, and the whiskers span the lowest to the highest observations. Comparisons were performed with the Kruskal-Wallis test followed by Dunn's post hoc test (n=7-10 per group). GCL—ganglion cell layer; INL—inner nuclear layer; IPL—inner plexiform layer; NFL—nerve fiber layer; ONL—outer nuclear layer; OPL—outer plexiform layer; RPE—retinal pigment epithelium;

FIGS. 12A-12D are a scheme of the experimental protocol (12A), images (12B) and box plots (12C, 12D) of a study assessing the effect of intravitreally administered 3K3A-APC on myeloid cells accumulation and CNV growth. 12B: Upper-view (upper panel) and depth Z-view (lower panel) color images of representative RPE-choroid specimens positioned with the RPE layer facing upwards and the choroid resting on the slide. Retinal blood vessels were stained green using fluorescein isothiocyanate (FITC)-dextran perfusion (green), and myeloid cells were stained red using anti-CD11b antibody and scanned using confocal microscopy from the RPE into the choroid. The left two stacked images (Control) are of an untreated eye (i.e., without any intervention); the 2 stacked in the middle (Laser) are of CNV-induced eye untreated with 3K3A-APC; and the 2 stacked images on the right (Laser+3K3A-APC) are of CNV-induced eye treated with 3K3A-APC. 12C and 12D: quantitative analysis of the total number of CD11b⁺ cells and of the CNV volume (μm³) and penetration depth (μm) of blood vessels, respectively, within the RPE-choroid specimens, presented as box-and-whiskers plot. The boxes span the 25th to the 75th percentile, the line inside each box denotes the median, and the whiskers span the lowest to the highest observations. Comparisons were performed with the Kruskal-Wallis test followed by Dunn's post hoc test (n=5-9 eyes per group);

FIGS. 13A-13B are images (13A) and a bar graph (13B) showing the effect of intravitreally administered 3K3A-APC on leakage from CNV following laser-induced eye damage. 13A: dynamic fluorescein angiography (FA) images of two eyes on days 4 and 11 post laser application (sequential real-time images were captured: “early”—the first minute from fluorescein injection, and “late”-every minute between 2 to 5 minutes following fluorescein injection). The cluster of 4 images on the left were taken 4 days after laser CNV-induction and before treatment with either saline or 3K3A-APC. On the 4^{th} and 7^{th} days post laser application, saline or 3K3A-APC were intravitreally injected and dynamic FA was conducted on the 11^{th} day. In the right cluster of 4 images, the upper images are of the saline-treated eye and lower ones are of the 3K3A-APC-treated eye. 13B: quantitative assessment of leaking lesions performed on day 11, comparing the percentage of leaking lesions in 3K3A-APC and vehicle (saline)-treated eyes. To account for two lesions per mouse, the data were analyzed using a generalized estimating equation, with the lesion status (“leaking” or “scar”) as a binary outcome and 3K3A-APC treatment (“no” or “yes”) as a between-subjects factor. The resulting p-value is presented;

FIGS. 14A-14C are images (14A, 14B) and a bar graph (14C) showing the neuroprotective effect of intravitreally administered 3K3A-APC in mice subjected to optic nerve crush (ONC). 14A: representative retinal flatmount images taken 14 days post-ONC (scale bar=20 μm). The left image (Control) is of a naïve eye (i.e., without any intervention); the middle image (Optic Nerve Crash) is of optic nerve crashed eye untreated with 3K3A-APC; and the right image (Optic Nerve Crash+3K3A-APC) is of optic nerve crashed eye treated with 3K3A-APC. Ganglion cell Layer (GCL) was stained pale blue using anti-RBPMS antibody. Blue staining is Hoechts staining of the nuclei. 14B: representative retinal cryosections images taken 14 days post-ONC (scale bar=100 μm). The left image is of a healthy retina (Control); the middle image is of a retina from an optic nerve-crashed eye not treated with 3K3A-APC (Optic Nerve Crash); and the right image is of a retina from an optic nerve-crashed eye treated with 3K3A-APC (Optic Nerve Crash+3K3A-APC). Ganglion cell Layer was stained pink using anti-RBPMS antibody. Blue staining is DAPI staining of the nuclei. 14C: quantitative assessment of total RGCs in retinal cryosections measured 14 days post ONC. A total number of cells and GCL layer length were measured using Image J. Data are presented as mean±SD and analyzed using one-way ANOVA followed by Tukey post hoc test (n=4-5 per group). GCL—ganglion cell layer; IPL—inner plexiform layer; INL—inner nuclear layer; OPL—outer plexiform layer; ONL—outer nuclear layer; and

FIG. 15 is a collection of images of representative retinal cryosections showing the effect of intravitreal administration of 3K3A-APC on microglial recruitment and activation in retina of mice subjected to optic nerve crush (ONC). Cryosections were taken 14 days post ONC. The left image is of a healthy retina (Control); the middle image is of retina from an optic nerve-crashed eye not treated with 3K3A-APC (Optic Nerve Crash); and the right image is of retina from an optic nerve-crashed eye treated with 3K3A-APC (Optic Nerve Crash+3K3A-APC). Microglia cells were stained green using anti-Iba1 antibody. Blue staining is DAPI staining of the nuclei. Scale bar represents 100 μm.

### DETAILED DESCRIPTION

The present disclosure is based on a discovery by the present inventors that 3K3A-APC, a variant (mutant) of APC, exhibited cytoprotective function in retinal diseases and acted as an anti-inflammatory and neuroprotective agent in the eye.

APC is a serine protease with several distinguishable biochemical activities. APC exerts anticoagulant activity by inactivating Factors Va and VIIIa, and it exerts cytoprotective and anti-inflammatory activities primarily through interactions with the endothelial protein C receptor (EPCR) and protease-activated receptor-1 (PAR-1) and PAR-3. To bypass its anticoagulant activities, several variants of wt-APC were designed. For example, Mosnier et al. (Mosnier et al., Blood, 104(6): p. 1740-1744, 2004) replaced three Lys residues with Ala residues in the wt-APC molecule, thus altering the factor Va binding exosites without modifying the exosites that recognize and bind to PAR-1 and PAR-3, thereby producing the 3K3A-APC variant with markedly reduced anticoagulant activity. The neuroprotective, endothelial-barrier protective and anti-inflammatory activities of 3K3A-APC were demonstrated in multiple models of neurological disorders (Griffin et al., Blood, 132(2):159-169, 2018; Griffin et al., Thromb Res, 141 Suppl 2:S62-64, 2016; Lyden et al., Front Neurol, 12:593582, 2021; Huuskonen et al., J Exp Med, 219(1), 2022). 3K3A-APC's neuroprotective effects manifest as protection of blood-brain-barrier (BBB) function, inhibition of neuroinflammation, inhibition of neuronal apoptosis, and regenerative effects targeting neuronal stem cells, and highlight the pleotropic cytoprotective activities of 3K3A-APC in the brain and neurodegenerative diseases. The safety of 3K3A-APC has been established (for example, by Lyden et al., Ann Neurol, 85(1):125-136, 2019).

The triad consisting of inflammation, blood-retinal-barrier disruption, and neuronal injury, characterizes most retinal pathologies that impair vision such as age-related macular degeneration and diabetic retinopathy. Currently, it is widely accepted that single-action-single-target agents are unlikely to fully treat these disorders. Hence, there is an unmet need for new pleiotropic agents that will impede inflammation, tighten the blood-retinal barrier (BRB) and induce neuroprotection, thereby preserving retinal function and vision in the long term. The present inventors envisioned 3K3A-APC as a new therapeutic candidate for retinal diseases, e.g., inflammatory retinal diseases, and neurologic damages.

To be noted, although 3K3A-APC has well-established anti-inflammatory, blood barrier protection and neuroprotective activities in the central nervous system CNS), its beneficial therapeutic effects in the eye in general, and in the retina in particular, could not have been apriori determined. Cell types in the nervous system exhibit a wide diversity in their morphologies, connectivity, and physiologies. This remarkable diversity endows the nervous system with the capacity for complex neuronal function. The retina is unique with individual cell types whose morphological and physiological properties are closely aligned with molecular signatures.

While both the blood-brain barrier (BBB) and the blood-retina barrier (BRB) serve to protect and maintain the respective tissues they enclose, they are distinct structures with differences in location, composition, permeability, and functions. At least the following key differences exist between the BBB and BRB: (i) The BBB is composed of endothelial cells connected by tight junctions; and (ii) the BRB consists of two components: the inner blood-retinal barrier, which is formed by tight junctions between the retinal capillary endothelial cells, and the outer blood-retinal barrier, formed by the retinal pigment epithelial cells. The outer barrier is unique to the retina and is very different from the BBB. Thus, for example, infiltration of cells of the immune system through the outer BRB has different features and nature than infiltration through the BBB. The present disclosure provides the effects of an APC variant on the outer BRB. These effects are unique to the eye and have no equivalents in the brain.

Furthermore, there is a variability between blood vessels located in different tissues and different vascular beds. Variability in the expression of growth factors, angiopoietins, Tie2, VEGF-R, EPCR, PAR and other cellular receptors, may result in variable response to APC variants. Moreover, the duration of APC exposure and APC clearance from the vitreous cavity may also modify the response. Thus, the retina has its own unique structure and function, and what is efficacious for the brain might not necessarily be efficacious in the retina.

For example, in contrast to the anti-angiogenic effect of APC on the development of choroidal blood vessels demonstrated by the present inventors, a proangiogenic effect of APC was demonstrated in brain ischemia models.

Based on experimental results obtained in an inflammation murine model, endotoxin-induced uveitis (EIU), which simulates intraocular inflammatory conditions as in uveitis, and further based on laser-induced CNV murine model, the present inventors successfully established the beneficial application of 3K3A-APC as a broad-spectrum cytoprotective molecule in various pathologies of the eye such as ocular inflammatory diseases and various retinal diseases and conditions.

### APC Variants

Embodiments disclosed herein pertain to the use of variants of APC in therapy of ocular pathologies. The term “APC variant”, as used herein, refers to any and all modified forms of wild type APC (wtAPC), including, but not limited to, a polymorphic variant, mutated APC or an interspecies homolog recombinant of wtAPC.

In some embodiments, the APC variant conserves substantially the same biological activities as wild-type APC. Variants conserving “substantially the same biological activities”, as referred to herein, are APC variants having about 60% identical, about 65% identical, about 70% identical, about 75% identical, about 80% identical, about 85% identical, about 90% identical, about 95% identical, or 100% identical biological activities as wild type human APC.

In some embodiments, the APC variant retains some of the biological activities of wtAPC but is impaired with respect to other biological activities of wtAPC. For example, an APC variant can retain the anti-apoptotic (cytoprotective), neuroprotective and/or barrier stabilizing activity, but its anti-coagulation activity is substantially impaired or even nulled.

As defined herein, a “functional fragment” is a partial sequence of the wild type APC protein or of a variant of APC that retains at least some of the biological activities of the whole or intact corresponding protein. For example, the functional fragment can comprise up to 95%, up to 90%, up to 85%, up to 80%, up to 75% or even less, of the whole protein, and it maintains substantially the same biological activities as that of the corresponding complete protein (i.e., wild type APC or APC variant).

In some embodiments, the APC variant is a derivative of the wtAPC or of a functional partial fraction thereof, in which the amino acid sequence has been modified post protein synthesis.

Post synthesis modification of APC or functional partial fragment thereof comprises chemical or physical modifications, or both, of one or more amino acids. APC or a functional partial fragment thereof that has undergone a chemical or physical modification is also termed herein “a chemical derivative” and “a physical derivative”, respectively. For example, a derivative of APC may have amino acid sequence which is identical to the wild type sequence, but contains a post-synthesis conformation modification (i.e., a physical derivatization).

Examples of APC derivatives useful for the purpose of embodiment described herein are further discussed and disclosed, for example, in U.S. Pat. No. 5,516,650.

In some embodiments, the APC variant is an APC mutant, in which one or more of the naturally coded amino acids has been substituted and/or deleted via post translation modification. “APC mutant” further includes the naturally coded amino acids sequence containing additional one or more amino acids. Post translation substitution modification comprises replacement of one or more naturally coded amino acids of APC with one or more amino acids selected from natural and non-natural amino acids. Post translation addition modification comprises addition of one or more amino acids selected from natural and non-natural amino acids to the naturally coded amino acid sequence. Modification resulting in substitution, addition or deletion of one or more amino acids is also referred to herein as “biological derivatization”.

The term “natural amino acid” as used herein refers to the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) as well as to pyrolysine and selenocysteine. “Non-natural amino acids”, as used herein, include, but are not limited to, amino acid analogs that function in a manner substantially similar to the naturally occurring amino acids. An amino acid analog, as referred to herein, is a compound that has the same basic chemical structure as a naturally occurring amino acid, for example, an α-carbon that is bound to a hydrogen, a carboxyl group, an amino group, and a residue R, however R is not a residue of any of the natural amino acids. For example, R may feature a chemical modification of a natural amino acid residue. Amino acid analogs further encompass modified peptide backbones, while still retaining the same basic chemical structure as a naturally occurring amino acid. Non-limiting examples of amino acid analogs include homoserine, norleucine, methionine sulfoxide, methionine methyl sulfonium.

The term “non-natural amino acid” further includes natural amino acids and amino acid analogs that have undergone chemical modifications. Non-limiting examples of chemically modified amino acids include N-acetylglucosaminyl-L-serine, N-acetylglucosaminyl-L-threonine, and O-phosphotyrosine.

Post-translation modification of the APC protein may include an “amino terminus modification group”, namely attachment of a molecule to the protein's terminal amine group. For example, terminus modification groups include polyethylene glycol or serum albumin. Terminus modification groups may be used to modify therapeutic characteristics of APC, including but not limited to increasing the serum half-life of APC.

A modified wild type APC can feature a chemical derivatization, physical derivatization, a biological derivatization or any combination thereof. In some embodiments, such derivatizations are regioselective.

The amino acid sequence of a chemically, physically or biologically modified wtAPC can be at least 70%, at least 80%, at least 85%, at least 90%, at least 95%, at least 98%, or even 100% identical to the wild type APC sequence.

Substitution of a single natural amino acid or a small percentage of natural amino acids in the encoded APC sequence is considered herein a “conservatively modified variant” of APC where the alteration results in the substitution of a natural amino acid or a small percentage of natural amino acids with chemically similar (analog) amino acid(s).

In some embodiments, an APC variant comprises a substitution, addition or deletion, or any combination thereof, of naturally coded amino acids, which provide one or more of the following features to the protein: increased affinity for a receptor, increased stability, modified (e.g., increased) aqueous solubility, increased solubility in a host cell, modulated protease resistance, modulated serum half-life, reduced anticoagulant activity, modulated immunogenicity, and/or modulated expression relative to the wild-type APC. “Modulating biological activity”, as used herein, refers to increasing or decreasing the reactivity and/or altering the selectivity, e.g., enhancing or decreasing the substrate selectivity of APC and any functional parts thereof.

In some embodiments, the biological activity of any of the APC derivatives is improved by about 5%, about 10%, about 15%, about 20%, about 30%, about 50%, about 60%, about 70%, about 80%, about 85%, about 90%, about 95%, about 100%, about 110%, or more, including any intermediate values therebetween, compared to the biological activity of any of the wtAPC or functional partial sequence thereof.

Usually, the modifications in wtAPC have beneficial effects such as improving its stability and/or its biological activity, and/or reducing or eliminating undesired activity, for example, reducing anticoagulant activity of APC, thereby reducing the risk of bleeding. For example, variants of recombinant APC may have markedly reduced anticoagulant activity but retained normal or near normal anti-apoptotic (cytoprotective) activity. In these variants, the ratio of anti-apoptotic to anticoagulant activity is greater than in wild-type or endogenous APC. Non-limiting examples of such recombinant APC mutants are: KKK191-193AAA-APC, also known as “3K3A-APC” (substitution of lysine residues 191, 192 and 193 with alanine residues in a surface-exposed loop containing Lys191-193); RR229/230AA-APC (substitution of arginine residues 229 and 230 with alanine residues); and RR229/230AA plus KKK191-193AAA-APC, a combination of 3K3A and RR229/230AA-APC also known in the art as “5A-APC” (see, U.S. Pat. Nos. 9,192,657 and 7,489,305). Given their reduced anticoagulant activity, these exemplary APC variants provide significantly reduced risk of bleeding (variants 5A-APC and 3K3A-APC have <10% residual anticoagulant activity). 3K3A-APC has been reported to provide neuroprotection and extended therapeutic window (Griffin et al., Blood, 2015, 125:2898-2907). Other APC mutants include APC-2Cys, K193E-APC, and E149A-APC disclosed in Griffin et al. (supra), and APC variants that include the substitution of residue 158 (Asp) with a non-acidic amino acid residue such as Ala, Ser, Thr or Gly, or a substitution of residue 154 (His) with an amino acid residue such as Lys, Arg or Leu.

In some embodiments, the APC variant is 3K3A-APC or a functional partial sequence thereof.

It is expected that during the life of a patent maturing from this application many relevant chemical, physical and biological derivatives of wtAPC or of functional partial sequences thereof will be developed and the scope of the term “activated protein C, functional partial sequence thereof, derivative thereof or variant thereof” is intended to include all such new technologies a priori.

### APC Variant-Based Ophthalmologic Therapies

### (i) Treatment of Ocular Inflammation

While the anti-inflammatory effects of APC and 3K3A-APC have long been appreciated in non-ocular tissues, disclosed herein, for the first time, is experimental evidence supporting the anti-inflammatory and neuroprotective activities of 3K3A-APC in the eye. The experimental results disclosed herein indicate a significant decrease in leukocytes migration into the retina following 3K3A-APC treatment, seen both by flow-cytometry and histological analysis. The predominant immune cells involved in the endotoxin-induced uveitis (EIU) model were neutrophils, followed by macrophages, which is concurrent with EIU's known inflammatory effects. Both immune cells subpopulations were similarly affected by 3K3A-APC's inhibitory actions in the retina (Example 3 herein).

The inhibitory effect of 3K3A-APC treatment on leukocytes, as shown herein (Examples 2-3), further extends to extravasation, namely, leakage of leukocytes through the capillary walls into surrounding tissues. Thus, it is shown herein that in addition to reduced leukocyte migration, 3K3A-APC treatment increased immune cell's retainment inside retinal blood vessels, as demonstrated by decreased numbers in the retinal parenchyma and increased co-localization to blood vessel walls. This protective effect could be attributed to APC's barrier stabilizing activities.

In one aspect, the present disclosure relates to a method for treating inflammation in the eye of a subject, the method comprises administering a therapeutically effective amount of a variant of APC or a functional partial sequence thereof.

An ocular inflammation treatable by a contemplated method may be inflammation that is associated with any known disease, disorder or condition, which may be an ocular or non-ocular disease, disorder or condition.

The term “inflammation associated with a disease, disorder or condition”, as used herein, includes inflammation caused directly by a disease, disorder or condition, and inflammation developed in a secondary stage or as a complication of a disease, disorder or condition whereby, optionally, the disease, disorder or condition worsen, exacerbates or progresses because of the inflammatory process. Ocular inflammation being featured as a synchronous or asynchronous sequela of a disease, disorder or condition, which may be ocular or non-ocular, is also regarded herein as ocular inflammation associated with a disease, disorder or condition. For example, an ocular or non-ocular disease which may be manifested as ocular inflammation is a systemic infectious or noninfectious disease.

In some embodiments, the inflammation treatable by a disclosed method is associated with an ocular or retinal disease, disorder or condition. The ocular inflammation may be a direct consequence of an ocular or retinal disease, disorder or condition, or manifest as a secondary stage or a complication an ocular or retinal disease, disorder or condition.

The ocular disease may be at least one of: anterior, intermediate and posterior uveitis, pan uveitis, endogenous and exogenous endophthalmitis, inflammatory disease of the optic nerve selected such as optic neuritis, papilledema, anterior and ischemic optic neuropath (AION), choroidal neovascularization (CNV), age-related macular degeneration (AMD), (wet AMD, dry AMD), pathologic or high myopia, pseudoxanthoma elasticum with angioid streaks, Beh et's disease, choroiditis, episcleritis, scleritis, thyroid ophthalmopathy and diabetes complications such as proliferative diabetic retinopathy (PDR) or non-proliferative diabetic retinopathy (NPDR).

The ocular disease may further be a neurodegenerative optic nerve or a retinal disease such as, but not limited to, retinitis pigmentosa, glaucoma, best Vitelliform macular degeneration (BVMD) or Stargard's disease.

In some embodiments, the ocular disease is anterior, intermediate and/or posterior uveitis, AMD or endophthalmitis.

“Inflammation associated with ocular conditions” as defined herein refer to accidental, occasional, one-time incidences in which inflammation develops as a result of, for example, traumatic injury, e.g., of the retina, complications during an ophthalmic medical procedure such as surgery, laser treatment or routine checkup, and the like.

The ocular disorder or condition may be at least one of: retinal leakage, blood vessel occlusion, oxidative damage, chronic inflammation, a traumatic injury such as, but not limited to, perforating and blunt trauma or post operative retinal detachment.

The ocular disorder or condition may further be some optics nerve disorders such as damage to the optic nerve and the death of associated retinal ganglion cells (RGCs) by elevated intraocular pressure (IOP), glaucoma, optic neuritis caused by multiple sclerosis, an autoimmune condition such as thyroid ophthalmopathy, sarcoidosis, or systemic lupus erythematosus (SLE), temporal arteritis (e.g., giant cell arteritis (GCA)), Lyme disease, viral infection such HIV or herpes, cat scratch fever (Bartonella), syphilis, and side effects of certain medications.

The ocular disorder or condition may further be bacterial and viral infections such exogenous or endogenous endophthalmitis.

Inflammasomes are innate immune system receptors and sensors that regulate the activation of caspase-1 and induce inflammation in response to microbial pathogens and foreign as well as host-derived danger signals. They have been implicated in a host of inflammatory disorders. The NLRP3 inflammasome is a multiprotein complex that plays a pivotal role in regulating inflammatory signaling. Upon activation, NLRP3 oligomerizes and activates caspase-1 which initiates the processing and release of pro-inflammatory cytokines IL-1p and IL-18 from their pro-IL precursors. NLRP3 is the most extensively studied inflammasome due to its array of activators and aberrant activation in several inflammatory diseases.

The NLRP3 inflammasome is constitutively expressed in various parts of the eye, including the retinal pigment epithelium, retinal microglia, Müller cells, astrocytes, conjunctiva, trabecular meshwork, and corneal epithelial cells. Accumulating evidence implicates that inflammasome activation contributes to tissue damage in various ocular diseases, including glaucoma, diabetic retinopathy, and AMD and an aberrant activation of the NLRP3 inflammasome was demonstrated in uveitis patients and murine models of uveitis. Previous studies have shown that wtAPC and 3K3A-APC can inhibit inflammasome activation in models of cardiac and renal ischemia-reperfusion injury via mTORC1 inhibition, thereby alleviating injury. The present inventors demonstrate in Examples 4 and 7 herein that in the retina, 3K3A-APC acts via inflammasome inhibition: in the inflamed retina, 3K3A-APC decreased both inflammasome priming (demonstrated by decreased NLRP3 levels) and its activation, through decreased IL-1β levels. With recent developments in anti-inflammasome therapies, 3K3A-APC is a promising NLRP3 inhibitor and may be beneficial in ocular pathologies such as AMD.

In some embodiments, the present disclosure relates to an anti-inflammasome therapy for treatment of an ocular pathology, the method comprises administering a therapeutically effective amount of a variant of APC or a functional partial sequence thereof, to a subject in need thereof, thereby treating the ocular pathology in the subject.

Ocular pathologies treatable by the disclosed anti-inflammasome therapy include any of the ocular diseases, disorders and/or conditions associated with activation of ocular NLRP3 inflammasome and manifest inflammation as a direct cause thereof, a secondary stage or a complication thereof and/or as a synchronous or asynchronous sequela thereof.

In some embodiments, the ocular pathology is AMD.

In some embodiment, the APC variant is 3K3A-APC.

In some embodiments, 3K3A-APC is administered to the subject via a systemic route, for example by IV injection.

In some embodiments, 3K3A-APC is locally administered to the subject via intravitreal injection and/or topical application of e.g., eye drops, ointment and the like.

### (ii) Cytoprotection

The retina is part of the central niveous system (CNS). Retinal ganglion cells (RGCs) are a specialized type of CNS neuron that gathers visual input and transfers it directly to the brain via the optic nerve. Neurodegenerative disorders of the retina involve optic nerve injury and the progressive death of RGCs. The death of RGCs is one of the main causes of irreversible blindness in the world.

APC signaling mediated by protease-activated receptors (PARs) is central to its cellular activities. In-vivo studies on a variety of acute and chronic neuropathologies have previously shown that PAR activation is required for APC neuroprotective actions in the CNS. In the neuro retina, PAR-1 expression was reported in retinal ganglion cells (RGC), Müller glial, rod photoreceptors (but not in cones), and the inner nuclear layer. The present inventor envisioned APC variants as promising multiple-action-multiple-target pleiotropic agents that may serve as an innovative therapeutic approach for cytoprotection in pathological ocular conditions. Indeed, the present inventors have successfully shown that the variant 3K3A-APC has neuroprotective activities. Example 12 herein demonstrates the ability 3K3A-APC to inhibit RGCs loss in a murine model of optic nerve crash (ONC).

Moreover, microglia have an increasingly recognized role in various retinal pathologies, including uveitis, AMD, diabetic retinopathy and optic nerve injury. The present inventors have demonstrated that 3K3A-APC treatment inhibits microglial recruitment, activation and translocation from the inner to the outer retina that was induced by ONC (Example 12 herein). These findings are critical for the therapeutic potential of 3K3A-APC to limit the progression of the inflammatory damage in neurodegenerative ocular diseases with vision loss.

In an aspect, the present disclosure relates to a method for ocular cytoprotection, the method comprises administering a therapeutically effective amount of a variant of APC, or a functional partial sequence thereof, thereby providing cytoprotection to the eye of the subject.

“Cytoprotection”, as used in the context of the present disclosure, refers to a process by which an APC variant (e.g., APC mutant) provides protection to ocular cells against harmful agents, which may be endogenous and/or exogeneous agents.

In some embodiments, the ocular cells protected by a contemplated method are neuronal cells. In the context of these embodiments, cytoprotection is actually neuroprotection.

Neuroprotection refers to a process or a strategy, which promotes protecting, salvaging, recovering, and preserving the integrity of neurons and neurovascular units (NVUs) to enable them to perform their physiological functions. Neuroprotection is also referred to as the prevention of neuronal cell death by intervening and inhibiting the pathologic processes that cause neuronal dysfunction and death. It is a disease-modifying event, a process that protects neurons and/or NVUs from deleterious effects at the subcellular, cellular, multicellular, or organismal level, caused by pathological insults such as, but not limited to, toxins, mechanical injuries or genetic mutations.

In some embodiment, the APC variant is 3K3A-APC.

In some embodiments, 3K3A-APC is administered to the subject via a systemic route, for example by IV injection.

In some embodiments, 3K3A-APC is locally administered to the subject, e.g., via intravitreal injection and/or topical application of e.g., eye drops, paste, spread, ointment and the like.

(iii) Treatment of a Macular Edema Associated Disease

Macular edema is defined as a collection of localized swelling in the macular area, leading to increased central retinal thickness. In the initial stage, the fluid accumulates in the outer plexiform layer (OPL) and/or the inner nuclear layer (INL). Swelling of the Müller cells has also been noted. The accumulated fluid may involve the extracellular retinal spaces. Macular edema is caused by pockets of fluid (usually leakage from damaged blood vessels) swelling up in the macula. It is a non-specific sign or sequelae for a myriad of intraocular and systemic diseases.

The main etiologies for macular edema include:

Diabetic macular edema (DME): vascular dysfunction is initially caused by the breakdown of the inner BRB, while the outer BRB is compromised in the later stages. The damage occurs, e.g., secondary to hyperglycemia (vascular endothelial cells are damaged due to their inability to regulate intracellular glucose levels), ischemia, enhanced reactive oxygen intermediates production, extracellular matrix degradation, and abnormal autoregulation (capillary basement membrane thickening).

Retinal vein occlusions: veins in the retina become blocked, and blood and fluid then leak out into the macula. The outer BRB is disrupted due to ischemia, raised hydrostatic pressure in the perifoveal capillaries, and a turbulent blood flow.

Coat disease: The inner BRB is disrupted due to damage to the endothelium of the retinal vasculature and abnormal pericytes. These abnormalities lead to multiple telangiectasias (sausage-like vessels) and retinal ischemia.

Retinal artery macroaneurysms (RAM): chronic hypertension, arteriosclerosis, and focal ischemia of blood vessel walls cause weakening of the blood vessel wall and subsequent aneurysmal dilatation.

Radiation retinopathy (RR): macular edema is the earliest feature of RR. The free radicals cause vascular damage (initially capillaries), leading to capillary nonperfusion. The pathogenies and clinical features are similar to diabetic retinopathy.

Hypertensive retinopathy (stage IV): the outer BRB is disrupted due to damage to the retinal vessels or ischemic hypoperfusion of the choroid, resulting in RPE damage.

Irvine-Gass syndrome: the surgical trauma during intraocular surgeries causes the breakdown of the blood-aqueous barrier via prostaglandin release. The inflammatory mediators subsequently diffuse into the vitreous cavity and also disrupt the BRB; this increases the permeability of the perifoveal capillaries. Macular edema may be a complication of glaucoma, retinal or cataract surgery.

Inflammatory diseases and disorders: inflammatory diseases and disorders of the immune system may affect the eye and cause swelling and breakdown of tissue in the macula, for example, cytomegalovirus infection, retinal necrosis, sarcoidosis, Beh et's syndrome, toxoplasmosis, Eales' disease, and Vogt-Koyanagi-Harada syndrome. Conditions like uveitis, where the body attacks its own tissues, can damage retinal blood vessels and cause swelling of the macula.

Non-arteritic anterior ischemic optic neuropathy (NAION): it is assumed that fluid can percolate into the subretinal and/or intraretinal spaces from the peripapillary choroid.

Edema after focal or panretinal laser burns (photocoagulation) (PRP): this occurs secondary to the inflammation induced during the procedure, combined with increases in macular blood flow secondary to the laser.

Drug-induced macular edema: topical epinephrine (antiglaucoma medication) causes the breakdown of the BRB that can lead to macular edema. Prolonged use of systemic tamoxifen can cause reversible macular edema. Systemic nicotinic acid disrupts the BRB by prostaglandins release and/or Müller cells toxicity (causes intracellular fluid accumulation). Topical latanoprost (antiglaucoma medication) causes blood-aqueous barrier disruption in early postoperative eyes.

Choroidal tumors: both benign and malignant tumors can lead to macular edema. Cystoid macular edema and subretinal fluid may be seen in tumors like choroidal melanoma secondary to infiltration of chronic inflammatory cells within the choroid adjacent to the tumor, and choroidal hemangioma due to the abnormal leaking vessels, respectively.

Retinitis pigmentosa (RP): the possible mechanisms responsible include the breakdown of the BRB due to the ‘toxic products’ released from the degenerating retinal cells, especially the RPE cells; failure of the RPE pumping mechanism, and muller cell dysfunction.

Age-related macular degeneration (AMD): with AMD, abnormal blood vessels leak fluid and cause macular swelling.

Macular pucker/vitreomacular traction (VMT): when vitreous in the aging eye or myopic eyes doesn't detach completely from the macula, the vitreous tugs on the macula or forms traction and pockets of fluid collect underneath it.

Hereditary/genetic disorders: retinoschisis or retinitis pigmentosa.

Injuries: trauma to the eye, all types of intraocular eye surgeries and Berlin's edema.

In an aspect, the present disclosure relates to a method for treatment of a macular edema associated disease or condition, the method comprises administering to a subject in need thereof a therapeutically effective amount of a variant of APC or a functional partial sequence thereof, thereby treating a macular edema associated disease or condition in the subject.

In some embodiments, the macular edema associated disease or condition is at least one of diabetic macular edema, retinal vein occlusion (RVO), postoperative cystoid macular edema or uveitis associated macular edema, age-related macular degeneration (AMD) or macular edema associated with an inflammatory disease or disorder of the immune system selected from cytomegalovirus infection, retinal necrosis, sarcoidosis, Beh et's syndrome, toxoplasmosis, Eales' disease or and Vogt-Koyanagi-Harada syndrome.

In some embodiment, the APV variant is 3K3A-APC.

In some embodiments, 3K3A-APC is administered to the subject via a systemic route, for example by IV injection.

In some embodiments, 3K3A-APC is locally administered to the subject via intravitreal injection and/or topical application of, e.g., eye drops, paste, spread, ointment and the like.

### (iv) Treatment of Ischemic Retinopathy

“Retinopathy”, as used herein, refers to any damage to the retina of the eyes, which may cause vision impairment. Retinopathy often refers to a retinal vascular disease, or damage to the retina caused by abnormal blood flow. Retinopathy, or retinal blood vessels related disease, can be broadly categorized into proliferative and non-proliferative types. Frequently, retinopathy is an ocular manifestation of systemic disease as seen in diabetes or hypertension. Diabetic retinopathy is the leading cause of blindness in working-aged people. It accounts for about 5% of blindness worldwide and is designated a priority eye disease by the World Health Organization.

Ischemia is a condition in which the blood flow (and thus oxygen) is restricted or reduced in a part of the body. The terms “ischemic retinopathy” and “retinal ischemia”, as used herein, are interchangeable and refer to retinopathy associated with ischemia.

In an aspect, the present disclosure relates to a method for treatment of ischemic retinopathy, comprising administering a therapeutically effective amount of a variant of APC or a functional partial sequence thereof to a subject in need thereof, thereby treating ischemic retinopathy in the subject.

Retinal ischemia is most often caused by other conditions that affects the retina. These include central retina vein occlusion (CRVO), branch artery or vein occlusion (BRVO), central or branch artery occlusion (CRAO, BRAO), retinal vasculitis infections or inflammation, retinopathy of prematurity (ROP) and diabetes. These conditions affect the blood flow into and out of the retina, which can lead to ischemia. In addition, any disease that damages blood vessels in the retina can cause macular edema.

The treatment for retinal ischemia will vary depending upon the cause. A common treatment is the use of anti-VEGF (anti-vascular endothelial growth factor) medicines, which help stop abnormal blood vessels from growing in the retina.

In some embodiments, the ischemic retinopathy is at least one of: central retinal artery occlusion (CRAO), branch retinal artery occlusion (BRAO), central retinal vein occlusion (CRVO), branch retinal vein occlusion (BRVO), diabetic retinopathy (DR), or retinopathy of prematurity.

In some embodiment, the APC variant is 3K3A-APC.

In some embodiments, 3K3A-APC is administered to the subject via a systemic route, for example by IV injection.

In some embodiments, 3K3A-APC is locally administered to the subject via intravitreal injection and/or topical application of, e.g., eye drops, paste, spread, ointment and the like.

### (v) Treatment of Choroidal Neovascularization Growth and Leakage

Choroidal neovascularization (CNV) is a common cause of vision impairment in patients with nAMD. Moreover, CNV can manifest as a secondary pathology of inherited and acquired conditions, including high myopia, angioid streaks, and hereditary, traumatic, or inflammatory disorders. Despite the effectiveness of anti-VEGF-based treatments, it is necessary to consider approaches that can inhibit the continuity of the neurodegenerative process and serve as an alternative for unresponsive patients. Given the multifactorial aspects that contribute to CNV development, a multi-target approach that simultaneously addresses multiple pathologic mechanisms may offer an effective strategy to achieve optimal therapeutic outcomes, which have not been fully achieved by solely targeting VEGF.

The present inventors have previously demonstrated that treatment with wtAPC effectively inhibited CNV development. Its protective effects in the retina are partially mediated through VEGF reduction and signaling via the Tie2 receptor. However, using wtAPC as a treatment for fragile and abnormal blood vessels associated with CNV may not be suitable due to its anticoagulant properties, which can increase the risk of ocular hemorrhage. The present inventors have further previously demonstrated that the APC variant 3K3A-APC maintains wtAPC's beneficial activities in the retina, as evidenced by the regression of CNV and reduced VEGF levels at the site of CNV.

Since inflammation is known to play a role in the pathogenesis of CNV development, it was hypothesized by the present inventors that anti-inflammatory activities of 3K3A-APC will contribute to CNV regression and could offer a potentially promising safe approach for treating CNV.

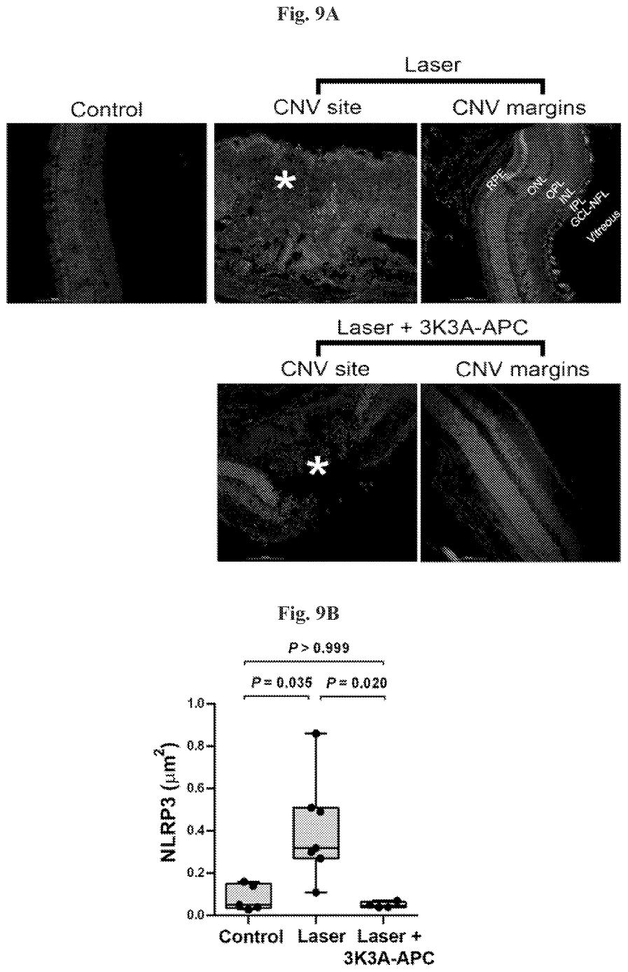

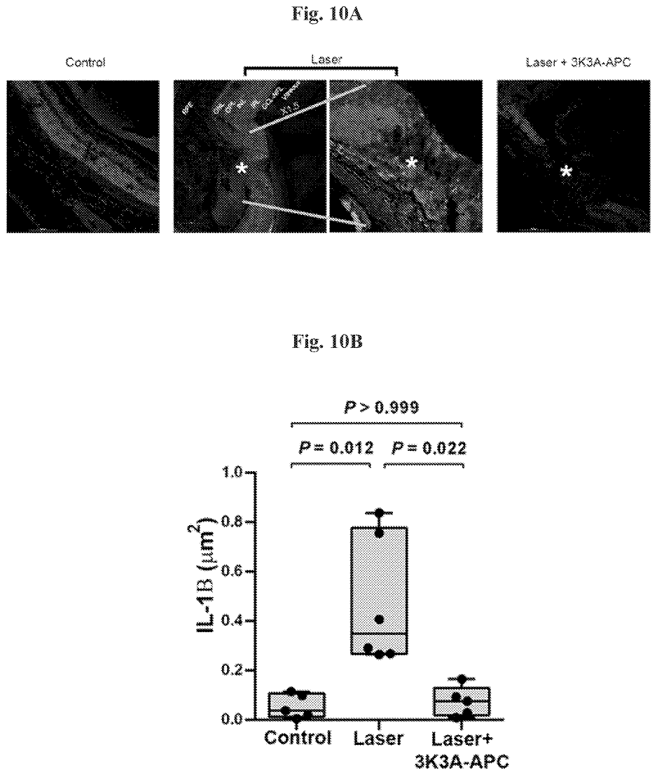

The pro-inflammatory protein inflammasome plays a crucial role in the innate immune response by sensing danger signals. Inflammasome activation occurs predominantly in activated macrophages and retinal microglia cells that infiltrate the site of CNV lesions. The present inventors have demonstrated that treatment with 3K3A-APC effectively reduced the levels of NLRP3 and its downstream effector, IL-1β, in the CNV lesion site and its surrounding regions following laser-induced CNV development (Examples 7 and 8 herein). It has been observed by the present inventors that significant increase in NLRP3 levels was at the CNV lesions, which suggest microglia as the source of NLRP3, but also in the RPE and the ganglion cell-nerve fiber layer (FIGS. 9-10 herein). It is still unclear which specific cell types in the retina 3K3A-APC acts on to limit inflammasome activation.

Microglia, the resident immune cells of the retina, are located in the nerve fiber layer, inner plexiform layer, and outer plexiform layer of the healthy eye, where they are closely associated with the vasculature, neuronal synapses, and glia. The present inventors observed an accumulation of amoeboid active microglia in the site of CNV lesion throughout the depth of the RPE-choroid and effected a significant reduction in microglial accumulation and a shift towards a more quiescent, ramified morphology upon 3K3A-APC treatment, as demonstrated in Example 9 herein.

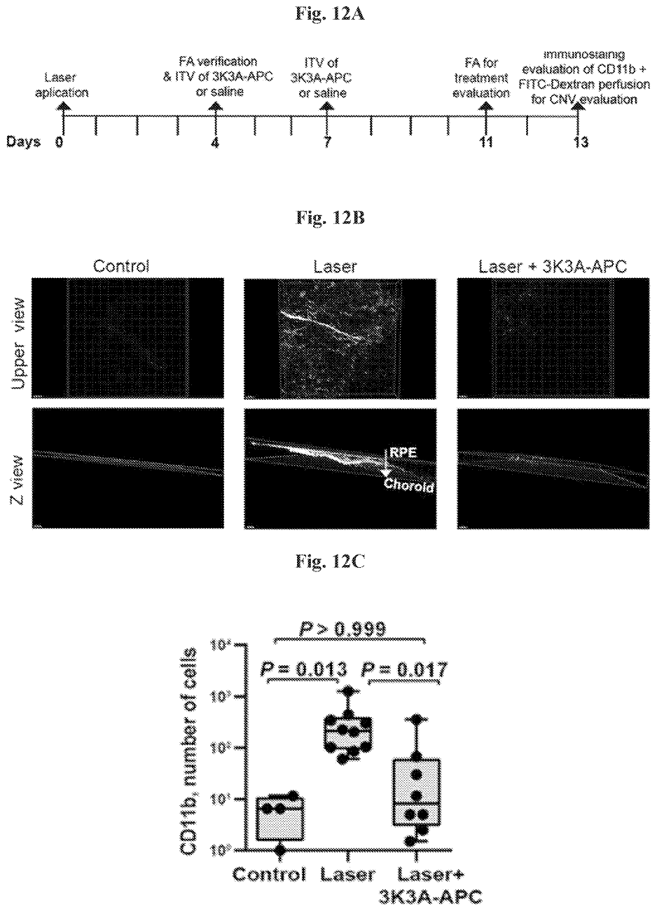

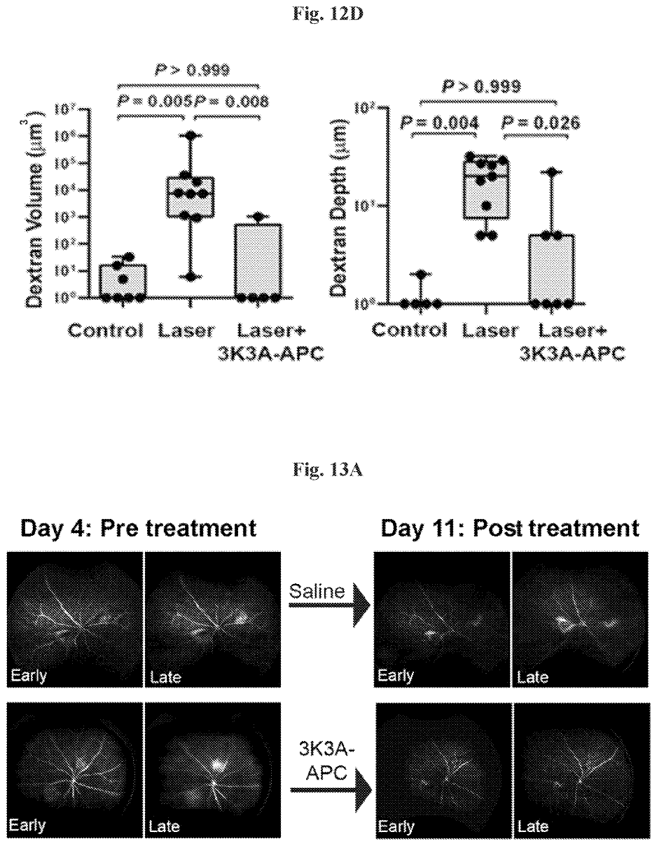

The increase in inflammatory cells in the CNV area can be attributed to the infiltration of circulating CD11b⁺ monocytes positive for various proangiogenic factors and inflammatory cytokines. It is demonstrated in Example 10 herein that 3K3A-APC treatment effectively reduced the accumulation of CD11b⁺ cells in the CNV area, even when administered during extensive inflammation (FIGS. 12A-12D). The ability of 3K3A-APC to inhibit myeloid cell extravasation of CD11b⁺ circulating monocytes out of retinal blood vessels into the retinal parenchyma, along with its endothelial blood barrier stabilization activities, can explain these observations.

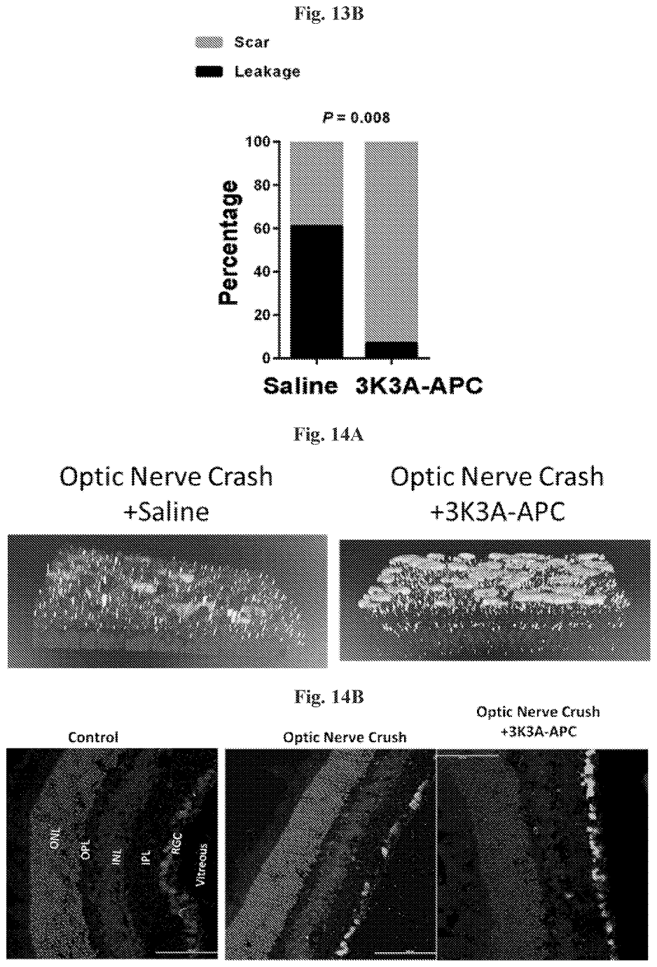

Given the high prevalence of leakage in patients with CNV, the present inventors evaluated the potential of 3K3A-APC treatment in halting CNV-associated leakage. Furthermore, looking ahead toward clinical application, the present inventors determined whether fluorescein angiography (FA) could serve as a surrogate marker for in-vivo evaluation of the efficacy of 3K3A-APC treatment. As described in Example 11 herein, the present inventors confirmed the presence of leakage from CNV and showed the efficacy of 3K3A-APC treatment by means of FA. These results demonstrate that 3K3A-APC effectively inhibits leakage from clinically active CNV, with a statistically significant increase in the odds of forming non-leaking lesions by 20.43 (95% Cl 2.19-190.50, p=0.008) with 3K3A-APC treatment (FIG. 13B). The current results are consistent with wt-APC-induced suppression of CNV leakage.

Given the complex and interconnected pathways involved in CNV development, the pleiotropic activities of 3K3A-APC offer a multi-target approach that may impede inflammation, defend BRB function, and protect the retina, thereby preserving vision. Furthermore, data disclosed herein suggest that 3K3A-APC holds promise as a therapeutic option for the treatment of CNV in patients with nAMD, considering its established clinical safety profile in treating severe ischemic stroke.

### APC Variant Administration Routes

In some embodiments, the APC variant or the functional partial sequence thereof are systemically administered to the subject. The systemic administration may be, for example, intravenous (IV) injection. This route of administration is demonstrated in Example 6 herein.

The terms “systemic administration” and “systemic delivery”, as used herein, are interchangeable and mean the administration of a drug product via a route that spreads the drug throughout the body of a subject, i.e., systemically. In systemic delivery, drugs and active agents are delivered directly into the bloodstream so as to reach and affect cells in all areas of the body. Systemic administration includes, for example, oral route and parenteral route (namely situated or occurring outside the intestine). Any route of drug administration other than oral is a parenteral route. The main parenteral routes of drug administration are intravenous (IV), intramuscular (IM), subcutaneous (SC) and intra-articular (IA), wherein the drug is usually administered via a hollow needle (i.e., injection), and topical administration. Injectable preparations are usually sterile solutions or suspension of a drug in water or other suitable physiologically acceptable vehicles. Volumes delivered can range from milliliter to liter quantities.

The time of onset of action for IV administration is seconds; for IM, SC, and IA injections, minutes. The bioavailability of a drug can be influenced by the location of the IM injection site. Parenteral dosage forms and delivery systems include injectables (i.e., solutions, suspensions, emulsions, and dry powders for reconstitution), intramammary infusions, intravaginal delivery systems, and implants. A solution for injection is a mixture of two or more components that form a single phase that is homogeneous down to the molecular level.

The terms “local administration” and “local application”, as used herein, are interchangeable and mean the administration of a drug product directly to the intended site of its action such that most of the administered drug product is confined within the treated site and exerts local effects due to the direct exposure thereto. Systemic absorption of drugs is minimal, hence systemic side effects can be avoided. Local administration includes, for example, topical application, wherein drugs are applied to skin/mucous membrane for local actions. Non-limiting examples for topical administration routes include skin, wherein drug is applied dermally (e.g., rubbing in of oil or ointment), or transdermally (through the skin); oral cavity (drugs may be delivered to oral mucosa in the form of lozenges or rinse); gastrointestinal tract (GIT), wherein nonabsorbable drugs can be used for exerting local effect only; rectum and anal canal, wherein drug in liquid/solid form is applied through this route for various local actions; eye, ear, and nose, wherein drugs can be delivered to nasal mucosa, eyes, or ear canal in the form of drops, ointments, and sprays. This route can be employed for allergic/infective and or inflammatory conditions of these organs; bronchi (inhalational), wherein drug is absorbed by bronchial mucosa through inhalation; vagina, wherein drugs can be applied/inserted in the form of tablet, cream, or pessary to vagina; and urethra, wherein drugs in the form of solution/jellies is applied to urethra.

Local administration into deeper areas for example, intra-ocular or intra-articular tissue can be effected by using syringe and needle. Non-limiting examples of local administration into deeper tissues of the eye include intravitreal injection, intra ocular-muscle administration, injection into the anterior chamber, injection into the suprachoroidal space, subconjunctival injection and intracorneal injection.

In some embodiments, the APC variant or the functional partial sequence thereof are intravenously injected to a subject afflicted with ocular inflammation.

In some embodiments, the APC variant or the functional partial sequence thereof are locally administered to the inflamed eye of a subject, for example, by intravitreal administration, e.g., injection, or by topical application of eye drops, paste, spread and/or ointment to the surface of the eye.

An intravitreal (into the vitreous cavity) injection is a way of delivering medications to the retina in the back of the eye. Drugs are administered into the eye by injection with a fine needle and are directly applied into the vitreous humor. Intravitreal injection is used to treat various eye diseases, such as AMD, diabetic retinopathy, and infections inside the eye such as endophthalmitis. Compared to topical administration, intravitreal administration is beneficial for a more localized delivery of medications to the targeted site, as the needle can directly pass through the anatomical wall of the eye (sclera).

The terms “therapy”, “treatment”, “treating”, “treat” as used herein are interchangeable and refer to: (a) preventing a disease, disorder, or condition from occurring in a human which may be predisposed to the disease, disorder and/or condition but has not yet been diagnosed as having it; (b) inhibiting the disease, disorder, or condition, i.e., arresting its development; (c) relieving, alleviating or ameliorating the disease, disorder, or condition, i.e., causing regression of the disease disorder and/or condition; and (d) curing the disease, disorder, or condition. In other words, the terms “therapy”, “treat,” “treatment,” and “treating,” extend to prophylaxis, namely, “prevent,” “prevention,” and “preventing,” as well as treatment per se of established conditions. Accordingly, use of the terms “prevent,” “prevention,” and “preventing,” would be an administration of the active agent to a person who has in the past suffered from the aforementioned conditions such as, for example, retinal inflammation or CNV, but is not suffering from the conditions at the moment of the composition's administration.

Thus, the terms “treatment”, “therapy” and the like include, but are not limited to, changes in the recipient's status. The changes can be either subjective or objective and can relate to features such as symptoms or signs of the disease, disorder or condition being treated. For example, if the patient notes improvement in visual acuity, reduced central visual field defects or decreased pain or pressure in the eye, then successful treatment has occurred. Similarly, if the clinician notes objective changes, such as by fluorescein angiography (FA), indocyanine green angiography (ICGA), optical coherence tomography (OCT) or OCT angiography (OCTA), then treatment has also been successful. Alternatively, the clinician may note a decrease in the size of lesions or other abnormalities upon examination of the patient (for example, grayish-white subretinal changes together with reduced retinal edema, hard exudations, reduced subretinal and intraretinal hemorrhage, reduced choroid neovascularization and reduced CNV leakage). This would also represent an improvement or a successful treatment. Preventing the deterioration of a recipient's status is also included by the term. Therapeutic benefit includes any of a number of subjective or objective factors indicating a desirable response of the condition being treated as discussed herein.

The term “therapeutically effective amount” as used herein, means the amount or dose of a compound, e.g., an APC variant that when administered to a subject for treating a disease, disorder or condition as defined herein, is sufficient to effect such treatment for the disease, disorder or condition. The therapeutically effective amount may sometimes be the lowest dose level that yields a therapeutic benefit to patients on average, or to a given percentage of patients. The ‘therapeutically effective amount’ can vary depending on the compound, the disease and its severity, and the age, weight, etc., of the subject to be treated.

In some embodiments, the method of treating or preventing ocular diseases, disorder or conditions, e.g., ocular inflammation, may include administrating to a subject in need thereof an effective amount of a variant of APC or a functional partial sequence thereof of from about 0.1 μg/μl to about 50 μg/μl, for example, from about 0.1 μg/μl to about 0.5 μg/μl, from about 0.1 μg/μl to about 1 μg/μl, from about 0.5 μg/μl to about 1 μg/μl, from about 0.5 μg/μl to about 2 μg/μl, from about 1 μg/μl to about 5 μg/μl, from about 2 μg/μl to about 8 μg/μl, from about 5 μg/μl to about 10 μg/μl, from about 11 μg/μl to about 15 μg/μl, from about 12 μg/μl to about 20 μg/μl, from about 15 μg/μl to about 20 μg/μl, from about 15 μg/μl to about 25 μg/μl, from about 20 μg/μl to about 28 μg/μl, from about 25 μg/μl to about 35 μg/μl, from about 10 μg/μl to about 40 μg/μl, from about 30 μg/μl to about 40 μg/μl, from about 35 μg/μl to about 45 μg/μl, or from about 40 μg/μl to about 50 μg/μl, including any subranges and individual values therebetween.

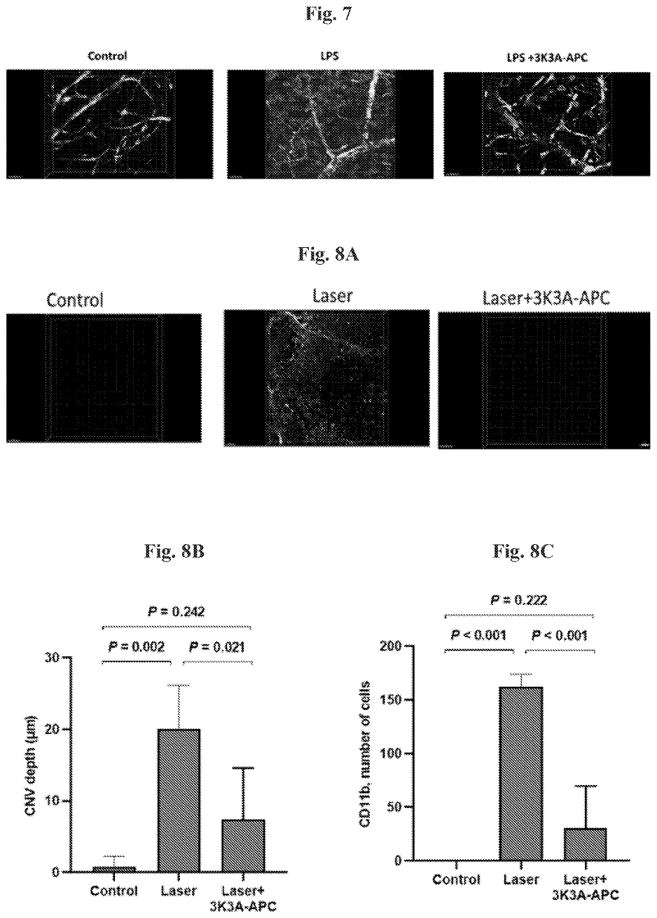

Intravenous administration of 3K3A-APC to mice and monkeys for 14 consecutive days at doses up to 5 mg/kg was well tolerated and did not result in any clinical signs of toxicity, effects on hematology or clinical chemistry parameters, or target organ toxicities following histopathological examination. In embodiments disclosed herein, 0.5 mg/kg or 0.2 mg/kg 3K3A-APC (see Examples 5 and 6, and FIGS. 7 and 8) was administrated intravenously, via tail vein, 10 min and 4 h after triggering the inflammatory response. In some embodiments,

In some embodiments, an effective amount of APC mutant or a functional partial sequence thereof, e.g., for treatment of humans is from about 50 μg/kg body weight to about 700 μg/kg body weight. For example, from about 80 μg to about 650 μg, from about 100 μg to about 600 μg, from about 200 μg to about 700 μg, from about 250 μg to about 600 μg, from about 300 μg to about 700 μg, from about 50 μg to about 70 μg, from about 60 μg to about 90 μg, from about 70 μg to about 100 μg, from about 50 μg to about 150 μg, from about 100 μg to about 150 μg, from about 200 μg to about 250 μg, from about 200 μg to about 400 μg, from about 350 μg to about 450 μg, from about 480 μg to about 550 μg, from about 400 μg to about 650 μg, or from about 500 μg to about 700 μg per kg of body weight, including any subranges and individual values therebetween.

In some embodiments, an effective amount of APC mutant or a functional partial sequence thereof is at least one of 120, 240, 360, or 540 μg/kg body weight.

The doses of, e.g., 3K3A-APC, applicable for IV administration may be determined based on studies known in the art. For example, the NeuroNEXT trial NN104 (RHAPSODY), a randomized, controlled, blinded dose-escalation safety trial for 3K3A-APC, evaluated the safety of ascending intravenous doses of 3K3A-APC in adult patients presenting acute ischemic stroke who were eligible for thrombolysis, thrombectomy, or both (Lyden et al., Ann Neurol., 85(1):125-136, 2019). Based on this pre-clinical work and a pilot Phase 1 trial, 4 dose levels of 3K3A-APC are selected in embodiments disclosed herein: 120, 240, 360 and 540 μg/kg. In some embodiments, 3K3A-APC is administered as a 100 mL intravenous infusion over 15 minutes every 12 hours (±1 hour) for 5 doses.

The method of treating any of the ocular pathologies described herein may benefit, for example generate a synergistic effect, when combined with other treatment modalities, particularly treatment modalities that address the prime motive or the trigger for development of a particular pathology. For example, a combined treatment protocol for treating inflammation in the eye may comprise the provision of an APC variant (systemically and/or locally) and other drug/active agent(s) for controlling the inflammatory process. Accordingly, systemic medications, for example, corticosteroids such as dexamethasone (e.g., Ozurdex) and derivatives thereof, with or without immunosuppressive agents may be indicated along with the APC variant. Additionally, or alternatively, therapies aimed directly at the neovascular process, such as any of the intravitreal anti-VEGF agents, are indicated, particularly when the anti-inflammatory therapy shows an insufficient response.

In some embodiments, APC variant is administered to a patient together with immunosuppressive agents, for example, an association of steroids, cyclosporine A and, in some cases, azathioprine. The steroids may be periocular or systemic steroids.

Since the inflammatory process is not only loco-regional and the whole immune system appears to be involved, the use of systemic steroids should also be considered. The safety and efficacy of immunosuppression are known. The choice of the immunosuppressant should be established based on the characteristics of the drug itself. For example, mycophenolate mofetil (MMF) can be the choice of drug for the long-term control of inflammatory CNV since it has proven to be effective in improving arteriolopathy and decreasing the amount of soluble mediators involved in CNV pathophysiology.

In some embodiments, APC variant is provided to a patient together with one or more treatments selected from anti-angiogenesis, anti-inflammatory, anti-bacterial, immunosuppressive, anti-platelet derived growth factor (PDGF), anti-fungal, anti-viral therapies and adeno-associated virus (AAV)-based gene therapy (AAV is a non-enveloped virus that can be engineered to safely deliver modified genetic material (e.g., DNA) to target cells and tissue impacted by otherwise difficult-to-treat conditions).

In some embodiments, the active agent(s) other than APC variant is one or more of: anti-VEGF drugs, steroids, angiopoietin-Tie2 signaling pathway drugs, bi-specific antibody targeting VEGF and Ang2, and NLRP3/IL1β inhibitors.

In some embodiments, the co-administered active agent or drug is administered together with APC variant in a single dosage form, optionally by intravitreal injection. Additionally, or alternatively, the co-administered active agent or drug is administered in one or more separate dosage forms, either before, simultaneously with, or subsequently after administration of APC variant. In some embodiments, the co-administered active agent is administered systemically.

Alternatively, or additionally the co-administered active agent is administered locally, optionally by intravitreal injection or topical application.

The regimen of APC variant administration in embodiments described herein, is dictated by various considerations such as the state of the retina, the progress of healing, tolerance of the patient and the like. For example, a single dose may be applied once a month or once a week for up to 6 to 8 weeks, wherein the gap between successive administrations and necessity of continuing APC variant administration is determined based on evaluation of the state of the treated eye, the progression of healing and the side effects.

The terms “comprises”, “comprising”, “includes”, “including”, “having” and their conjugates mean “including but not limited to”.

The term “consisting of” means “including and limited to”. As used herein, the singular forms “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects as delineated hereinabove and as claimed in the claims section below find experimental support in the following examples.

### EXAMPLES

Reference is now made to the following examples, which together with the above descriptions illustrate some embodiments in a non-limiting fashion.

### Materials and Methods

### (i) Endotoxin-Induced Uveitis Animal Model and Intravitreal Injections

Uveitis represents a diverse array of intraocular inflammatory conditions that can be associated with complications from autoimmune diseases, bacterial infections, viral infections and/or chemical injuries, and constitutes 10-15% of all cases of blindness. Endotoxin-induced uveitis (EIU) using lipopolysaccharide (LPS) is an established animal model for ocular inflammation. In this model, an increase in inflammatory mediators leads to blood retinal barrier (BRB) breakdown, leukocyte influx, and retinal edema (Klaska and Forrester, Curr Pharm Des, 21(18):2453-67, 2015; Rosenbaum et al., Nature, 286(5773):611-613, 1980). This valuable model of non-autoimmune human anterior uveitis and panuveitis served the present inventors in determining the role of APC variants such as 3K3A-APC in ocular inflammation processes. EIU allowed the direct investigation of innate immunity driven inflammation in the eye and studies of the effect of anti-inflammatory therapies disclosed herein.

Male C57BL/6J mice, 8-10-week-old and weighing 19 to 25 grams were purchased from Envigo (RMS, Israel). All animal experiments were performed according to the ARVO statement's guidelines for the Use of Animals in Ophthalmic and Visual Research and the approval of the Institutional Animal Care and Use Committee at Rabin Medical Center. Mice were randomized to either 3K3A-APC pretreatment or vehicle pretreatment. The endotoxin (LPS) was injected either systemically or intravitreally and induced a robust innate immune response in the retina and vitreous, as well as in the anterior chamber of the eye.

Intravitreal injection of 3K3A-APC followed by experimental uveitis induction was conducted as previously described (Healy et al., J Thromb Haemost, 2021, 19(1): p. 269-280; Coyle et al., Biomolecules, 2021, 11(8)). Briefly, 1 μl murine recombinant 3K3A-APC (Scripps Research, USA) or glycerol solution were prepared and injected 1 hour or 4 hours post LPS administration. Intravitreal (ITV) 3K3A-APC concentration of 1 μg/μl/eye, was chosen based on previously performed dose-dependent analysis of intravitreal injection of wt-APC, and a following study comparing ITV injection of wt-APC and 3K3A-APC (Livnat T. et al., Biomolecules, 11:1-13, 2021; Livnat T. et al., Exp. Eye Res., 186:107695, 2019).

Next, 1 μl of 250 ng LPS from Escherichia coli (Sigma-Aldrich, MO, USA) diluted in saline was injected to the right eye of anesthetized mice using a microsyringe. A third group injected with 3K3A-APC followed by saline served as additional control. 3K3A-APC and LPS were injected intravitreally under an operating microscope (Zeiss Opmi 6S Microscope; Carl Zeiss Microscopy GmbH, Germany), using a microsyringe (33-gauge; Hamilton, USA).

Animals were anesthetized with intraperitoneal (IP) injection of ketamine 100 mg/kg and xylazine 10 mg/kg. For all animal experiments, animal allocation to treatments was randomized, and each experiment was repeated 2-3 times.

### (ii) Vascular Imaging and Flatmount Immunostaining

The concept of topographic mapping is central for understanding the visual system at many levels. Retinal maps were obtained based on flatmount preparations. Dissection and relaxing cuts that flattened retina isolated from mice eyes were applied, which rendered the quasi-spherical retina into a 2D preparation. Specifically, a retina flatmount was prepared by placing the retina onto a slide with the eyecup facing up and cutting the retina in each quarter with a micro-scissor. After excess flatmount solution was removed, the eyecup was spread open like a flower.

Twenty-four hours post-EIU induction (peak of inflammation in the EIU model), or 4- and 13-days post laser application (in the laser-induced CNV model) mice were anesthetized, and 0.1 ml of 25 mg/ml fluorescein isothiocyanate (FITC) dextran conjugate (MW 500k, Sigma-Aldrich, MO, USA) was injected into the left ventricle of the mouse heart. Five minutes later, mice were sacrificed, and a flatmount specimen of sensory retina or the choroid were separated from the eyecup and flattened on slides. Flatmount specimens were fixed in 4% para formaldehyde (PFA) for 10 minutes. Slides were incubated in phosphate-buffered saline (PBS)-Triton™×100 0.5% solution at 4 °C overnight and later blocked for 2 hours at room temperature (RT) in 5% normal donkey serum (NDS; Sigma Aldrich, Israel). Slides were incubated overnight 4 °C with the first antibody diluted in blocker: rat anti-mouse CD11b antibody (1:200; Abcam, UK), rabbit anti-mouse Iba1 antibody (1:100 WAKO, Japan) or rabbit anti-mouse antibody VEGFA (1:400; Abca, UK). The secondary antibody was incubated at 4 °C overnight: Alexa Fluor® 568 conjugated goat anti-rat IgG or Alexa Fluor© 568 conjugated goat anti-rabbit IgG, respectively (1:100; Invitrogen, Waltham, MA, USA). The slides were covered with an anti-fade reagent (Invitrogen, MA, USA). A specimen incubated with non-immune serum was used as a staining control.

For choroidal thickness measurement, the Z axis of two images (one spanning the optic nerve and the other at a distal end) were measured at the center of each image and averaged. Volume of CD11b, Iba1 and VEGF staining was measured using Imaris x 64 7.1.1 software (Oxford Instruments, High Wycombe, UK). CD11b cell count was performed using Imaris software, modelling each CD11b cell to a sphere, and counting the sphere number.

(iii) Cryosection Histology and Immunofluorescence Staining

Cryosections, also known as frozen section biopsies, are rapidly and relatively easily prepared prior to fixation, and provide a good visualization of fine details of the cell. Cryosections are superior to paraffin- or resin-embedded sections for the preservation of antigenicity and detection of antigens by microscopy. In general, the sample is frozen quickly in either isopentane or liquid nitrogen. Rapid freezing reduces ice crystal formation and minimizes morphological damage. The preparation of cryosections does not involve the dehydration steps typical of other sectioning methods, and sectioning, labeling, and observation of specimens can usually be carried out in one day.

Twenty-four hours post-EIU, or 4 days post laser induced CNV, mice were sacrificed, and eyes were removed, punched with a 30 g needle, and fixed in 4% PFA for 2 hours at RT. Eyes were washed with increasing concentrations of sucrose in PBS and incubated with a final concentration of 30% sucrose overnight at 4 °C. Eyes were then embedded in Tissue-Tek® O.C.T.™ Compound (Sakura Finetek, Japan) on dry ice and kept at −80 °C (this compound is a formulation of water-soluble glycols and resins, providing a convenient specimen matrix for cryostat sectioning at temperatures of −10 °C and below). Serial sections of 10 μm thickness were cut using a cryostat (Leica Biosystems, Germany).

In EIU studies, sequential cryosections of each eye were stained with anti-CD11b (Abcam, UK) and anti-Iba1 (WAKO, Japan) antibodies as follows: cryosections were blocked with 10% NDS for 1 hour at RT and then incubated with rat anti-mouse CD11b antibody (1:200) or rabbit anti-mouse Iba1 antibody (2 μg) at 4 °C overnight. The next day, sections were incubated with Alexa Fluor® 568 conjugated goat anti-rat IgG or Alexa Fluor® 488 conjugated donkey anti-rabbit secondary antibodies (1:100, Invitrogen, USA). Nuclei were counterstained with of the nucleic acid dye 4′,6-Diamidino-2-phenylindole dihydrochloride (DAPI) (NucBlue™ Fixed Cell stain, Molecular Probes, USA). Images were captured using a fluorescence microscope (Axio Imager.Z2, Carl Zeiss Microscopy GmbH, Germany).

In CNV lesion studies, sequential cryosections of each eye from the lesion area were blocked with 3% NDS for 2 hours at room temperature and then incubated with rabbit anti-mouse Iba1 antibody (WAKO, Japan) (2 μg), rabbit anti-human IL-1β antibody (1:25, Abcam, UK) or rabbit anti-mouse NLRP3 antibody (1:200, Abcam, UK) at 4 °C overnight. The next day, sections were incubated with Alexa Fluor 488 conjugated donkey anti-rabbit secondary antibody (1:100, Invitrogen, USA). Nuclei were counterstained with DAPI (NucBlue™ fixed cell stain, Molecular Probes, USA). Images were captured using a fluorescence microscope (Nikon AX confocal system, Nikon, Tokyo, Japan) under the same conditions. Area of staining was measured using ImageJ software (NIH, USA).

### (iv) Retinal Dissociation and Flow-Cytometry Analysis

A total of 6-7 retinas were pooled to obtain >1 million cells for flow-cytometry. Retinas were isolated as previously described (Chu et al., Dis Model Mech, 2016, 9(4): p. 473-481). Briefly, retinas were removed in whole from the intact eye, placed in 1 ml of ice-cold serum-free RPMI (Biological Industries, Israel) and then dissected into small pieces. Retinas were further enzymatically digested in 1 mg/mL Collagenase B (Roche, Switzerland) and 0.5 mg/mL DNases 1 (Sigma-Aldrich, MA, USA; an endonuclease that digests single- and double-stranded DNA) for three 10-minute cycles in a rotating water bath warmed to 37 °C. The cell suspension was filtered through a 70 μM mesh to remove debris, centrifuged for 5 minutes (×400 g, 4 °C) and the pellet was re-suspended in FACS buffer and counted for live cells using Trypan Blue (Sigma-Aldrich, MA, USA). Cells were stained with Allophycocyanin-conjugated CD45, Phycoerythrin (PE)-CD11b, Cyanine 7 (Cy7)-Ly6C and Brilliant Violet (BV) 421-Ly6G (BioLegend, USA) according to manufacturer's protocol. Matched isotype controls were used in a similar manner. Labeled cells (0.5×10⁶) from each sample were acquired and analyzed using the Gallios™ 10-channel flow-cytometer and Kaluza Analysis Software (Beckman Coulter, IN, USA).

### (v) In Vivo Laser-Induced Choroidal Neovascularization (CNV) Animal Model

Choroidal neovascularization was induced based on Weinberger et al., 2017 (Weinberger D. et al., Curr. Eye Res., 42:1545-1551, 2017). Briefly, diode laser indirect ophthalmoscope (Iris Medical Oculight SLX System©, Iridex, Mountain View, CA, USA) was used with a laser power of 350 mW for a duration of 100 msec, and a condensing lens of 90 diopters. Two laser applications were applied to the right eyes, at a distance of 1 to 2 optic disc diameters around the optic nerve. Disruption of the Bruch's membrane was identified by the appearance of a white bubble at the site of photocoagulation.

Animals were anesthetized with intraperitoneal (IP) injection of ketamine 100 mg/kg and xylazine 10 mg/kg. Mice injected with vehicle (50% glycerin diluted in saline), with or without laser application, served as controls. For all animal experiments, animal allocation to treatments was randomized, and each experiment was repeated 2-3 times.

### (vi) Fluorescein Angiography (FA)

After mice were anesthetized, their pupils were dilated using tropicamide 0.5% (Fischer pharmaceutical labs, Israel), and 0.1 ml 2.5% fluorescein sodium (Novartis, Switzerland) was injected intraperitoneally (IP). Sequential real-time photos were captured during the early phase (namely, during the first minute from fluorescein injection) and late phase (every minute between 2 to 5 minutes following fluorescein injection). Color fundus photographs and fluorescein angiography (FA) images were taken using the Optos® California UWF imaging system (Optos® Inc., USA). Two masked retina specialists evaluated the fluorescein angiograms and sorted each laser spot with “leakage,” when hyperfluorescent lesions showed blurred margins increasing in size over time, and “no leakage” or “scar” when hyperfluorescent lesions showed distinct margins with no blur of margins over time.

(vii) Statistical Analysis

Statistical analysis was performed using GraphPad Prism version 9.3.1 for Windows (GraphPad Software, San Diego, CA, USA) or SPSS version 26 (IBM Corp., Armonk, NY, USA). Continuous variables were presented as the median and interquartile range (IQR).

In microscopic imaging studies, the values of four microscopic fields (two slides for an eye, two microscopic fields for a slide) obtained from an eye were averaged into one value and used as raw data for further analysis. The data were presented as mean±standard deviation (SD) and analyzed using one-way ANOVA followed by Tukey's post hoc test or unpaired two-tailed Student's t-test as indicated. In flow cytometry studies, the data were presented as mean±SD and, to account for inter-experimental variability, analyzed using mixed-effects models, with treatment as a between-subjects factor and experiment as a within-subjects factor, followed by Tukey's post hoc test. Differences were considered statistically significant if the P value was less than 0.05.

In laser induced CNV studies, categorical variables were presented as counts, proportions, and/or percentages. To evaluate the effect of 3K3A-APC treatment on NLRP3 and IL-1β levels, the number of Iba⁺ or CD11b⁺ cells at the CNV sites, the total CNV volume, and the penetration depth of blood vessels, the Kruskal-Wallis test was used followed by Dunn's post hoc test. To evaluate the effect of 3K3A-APC on the laser lesion status taking into consideration that there were two laser lesions per mouse, a generalized estimating equation was used, with the lesion status (“leaking” or “non-leaking”) as a binary outcome and 3K3A-APC treatment (“no” or “yes”) as a between-subjects factor. To evaluate the effect of laser lesion status on VEGF volume taking into consideration that there were two laser lesions per mouse, a linear mixed model was used, with the VEGF volume as a continuous dependent variable and the lesion status (“leaking” or “non-leaking”) as an independent variable. Differences were considered statistically significant if the p value was less than 0.05.

Data were graphically displayed using the box-and-whisker plots, which describes the behavior of the data in the middle as well as at the ends of a distribution. Box plots divide the data into 3 equally sized intervals called quartiles or, alternatively, into four equal sized segments or percentiles, each containing exactly a quarter (25%) of the data.

The “box”, using the 1^{st} and 3^{rd} quartiles, shows how individual values are grouped around a value of central tendency—the median (the 50th-percentile), meaning that exactly 50% of the data is at or above than this value (and exactly half is at or below). The median is also designated as Quartile 2 or Q2. The 1^{st} quartile (Quartile 1 or Q1) is the 25th-percentile and represents a value wherein 25% of the data is less than this value. The 3^{rd} quartile (Quartile 3 or Q3) is the 75th-percentile and represents a value wherein 25% of the data is greater than this value (or 75% of the data is below that point). The “whiskers” define the extreme edges of the distribution as defined by values that are 1.5 rimes the interquartile range. Values outside the whiskers are “outliers” or values that are unusual given the shape of the distribution.

The interquartile range (IQR) is the width of the box in the box-and-whisker plot. That is, IQR=Quartile 3−Quartile 1. The IQR can be used as a measure of how spread out the values are. If a data value is very far away from the quartiles (either much less than Quartile 1 or much greater than Quartile 3), it is sometimes designated as outlier. The accepted definition for an outlier is a number which is less than Quartile 1 or greater than Quartile 3 by more than 1.5 times the interquartile range.

### Example 1

### The Effect of 3K3A-APC on Leukocyte Number and Extravasation in Endotoxin-Induced Uveitis (EIU)

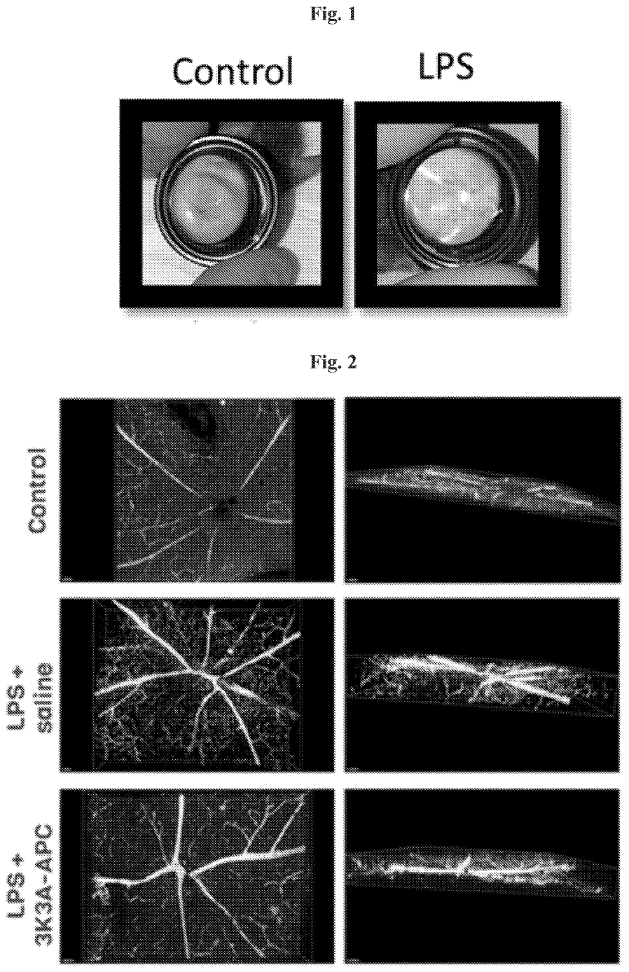

To determine the effect of endotoxin-induced uveitis (EIU), an in-vivo clinical evaluation of mice eyes was performed by indirect ophthalmoscopy at several time-points post LPS injection. In concurrence with previous reports, it was found that the peak of the inflammatory response was 24 h post-EIU induction, assessed by the amount of vitreous haze. Representative fundus images of naïve eye and LPS injected eye, 24 h post-EIU induction are presented in FIG. 1.

As seen in FIG. 1, naïve eyes demonstrated a normal fundus with clean vitreous, whereas 24 hours post-EIU induction, a notable vitreal haze (vitritis) was noticed, partially obscuring the optic disc. Most of the immune reaction was in the posterior segment of the eye, in the vitreous and retina, defining the EIU model as posterior uveitis.

CD11b belongs to the a subunits of the integrin receptor family and acts as a transmembrane molecule, critical in cellular adhesion and signal transduction. Integrins mediate multiple cellular functions, including adhesion, migration, complement binding, and cell survival. In immunophenotyping, CD11b is a leukocyte-specific receptor and is regarded as a marker for monocyte/macrophages, granulocytes, neutrophils, and natural killer cells. Functionally, CD11b regulates leukocyte adhesion and migration to mediate the inflammatory response.

Retinal blood vessels were fluorescently marked by in-vivo injection of fluorescein isothiocyanate (FITC)-dextran 24 hours post-EIU induction, with and without 3K3A-APC treatment. Immediately thereafter, retinas were isolated and prepared as flatmounts. Additional immunostaining for CD11b allowed the detection of myeloid cells inside blood vessels and in retinal parenchyma.

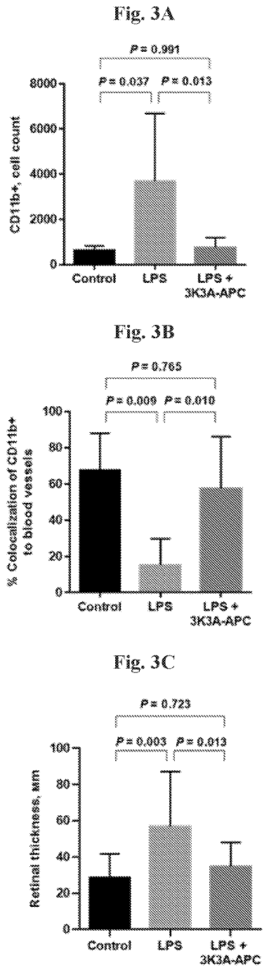

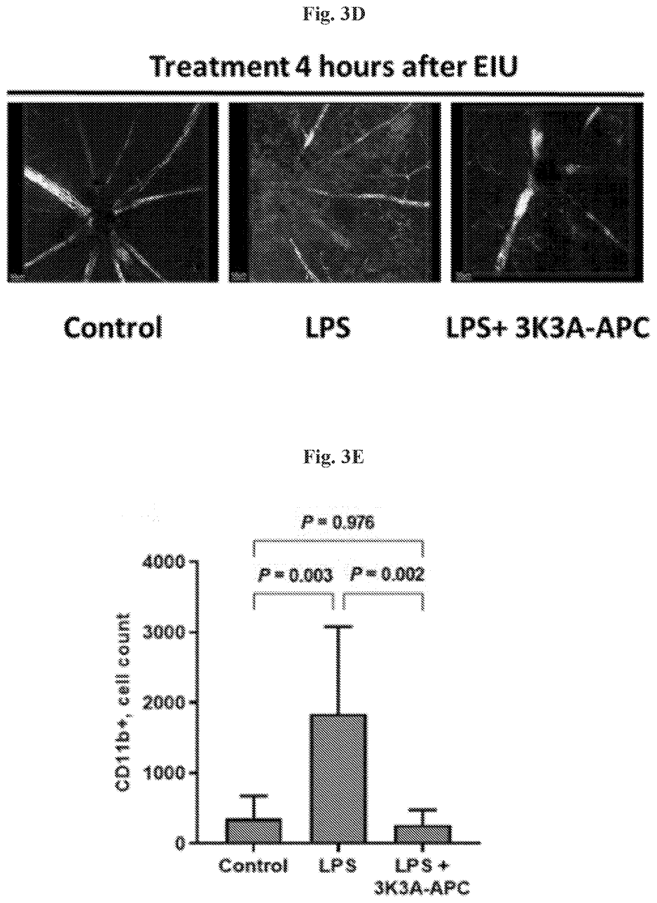

Representative color images of CD11b staining (red) and blood vessels staining (green) in flatmounts of control, EIU (LPS only) and LPS+3K3A treated eyes are shown in FIG. 2. Quantitative measurements are presented in FIGS. 3A-3C. The bars in FIG. 3A represent the CD11b positive cells count in the retinal parenchyma. As shown, LPS significantly increased the overall number of CD11b positive cells in the retina compared with control eyes (3968±2984 vs 629±202 cells; p<0.05). 3K3A-APC pretreatment significantly reduced CD11b positive cells numbers compared with LPS alone, to a level almost similar to controls (770±418 vs. 3968±2984; p<0.05). When analyzing co-localization of CD11b positive cells to dextran-stained vessels, an increased number of CD11b positive cells colocalized in retinal vessels was detected in 3K3A-APC pretreated eyes compared with LPS alone (58±29 vs. 15±14; p<0.05; FIG. 3B), similarly to controls which also demonstrated significantly higher numbers of leukocytes cells retained in the retinal blood vessels (68±20 vs. 15±14, p<0.01). This indicated that the anti-inflammatory effect of 3K3A-APC is associated with both decreased extravasation and higher retention within retinal blood vessels.

Retinal thickness is another morphologic marker for inflammation and is used to assess disease activity in humans. Quantitative analysis of blood vessel depth demonstrated a significant increase in vessel volume in the LPS group compared with controls (57±30 μm vs. 29±13 μm; p<0.05), indicating an increase in retinal thickness in eyes treated with LPS. Pretreatment with 3K3A-APC, however, resulted in a much smaller increase in retinal thickness compared with LPS only eyes (35±13 μm vs 57±30 μm; p<0.05; FIG. 3C), further indicating a successful reduction in retinal inflammation which may be of clinical relevance.

To further evaluate the potential clinical use of 3K3A-APC, a clinical situation wherein anti-inflammatory treatment is given to patients when retinal inflammation already exists, was simulated. For this purpose, EIU was induced with ITV injection of LPS, and four hours later eyes were treated with either 3K3A-APC or vehicle. Retinal flatmounts were prepared as described above. In eyes treated with 3K3A-APC four hours post-LPS, a dramatic reduction in CD11b staining was visible, indicating that 3K3A-APC treatment applied to existing inflammation still succeeded in reducing myeloid cell numbers in the retina (FIG. 3D). CD11b⁺ cell numbers markedly increased after LPS injection (347±327 cells vs. 1837±1240 cells in controls vs. LPS, respectively; p=0.003; FIG. 3E), however, 3K3A-APC treatment significantly reduced their numbers, to a level similar to that in the controls (262±217 cells vs. 1837±1240 cells; p=0.002, FIG. 3E).

### Example 2

### The Effect of 3K3A-APC on Retinal Leukocyte Percentage in EIU

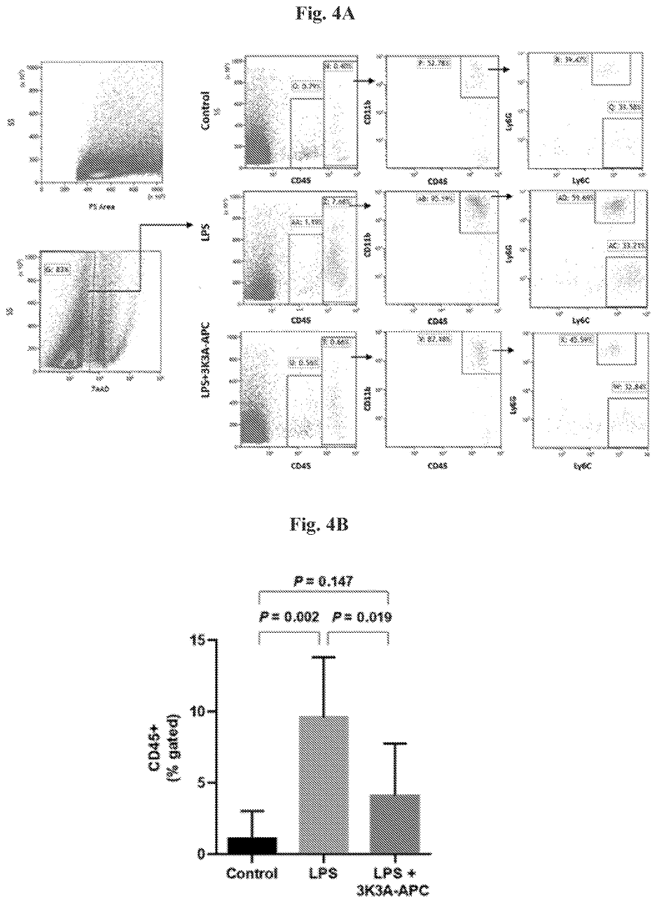

To further assess the anti-inflammatory potential of 3K3A-APC, its effect on leukocyte recruitment and leukocyte cells subpopulations (e.g., macrophages, neutrophils and microglia) in the eye following EIU, was determined using flow-cytometry. Twenty-four hours after intravitreal injections, single cell preparations (prepared as described in Materials and Methods) were analyzed using flow-cytometry in all treatment groups: (1) LPS; (ii) LPS+3K3A-APC; and (iii) control (no treatment at all). The results are presented in FIGS. 4A-4B.

The gating strategy used excluded debris and dead cells using 7AAD negative staining (FIG. 4A, two vertical dot plots on the left). Control eyes were largely devoid of leukocytes apart from a population of CD45 low CD11b low (CD45⁻CD11b⁻) non-inflammatory cells that were Ly6G⁻ and Ly6C⁻ representing resident retinal microglia (FIG. 4A, upper panel). CD45 is an antigen found on the surface of all nucleated hematopoietic cells, except for mature erythrocytes. A given cell is said to be “CD45 positive” if an isoform of the CD45 antigen is present on its surface.

Ly6 (known as lymphocyte antigen 6 or urokinase-type plasminogen activator receptor (uPAR)) is family of proteins that share a common structure but differ in their tissue expression patterns and function. A total of 35 human and 61 mouse Ly6 family members have been identified. Depending on which tissues they are expressed in, Ly6 family members have different roles. For example, they are involved in cell proliferation, cell migration, cell-cell interactions, immune cell maturation, macrophage activation, and cytokine production. Many Ly6 proteins are expressed in a lineage-specific fashion, and their expression often correlates with stages of differentiation. As such, Ly6 proteins are used as surface markers for leukocyte subset identification. Murine neutrophils display prominent surface expression of several Ly6 proteins, including Ly6B, Ly6C, and Ly6G. For most Ly6 proteins, a role in neutrophil functions, such as migration, is recognized. Ly6G (lymphocyte antigen 6 complex locus G6D) is expressed by myeloid-derived cells in a tightly developmentally regulated manner in the bone marrow. Ly6G is a good marker for detection of peripheral neutrophils, monocytes and granulocytes. Ly6C is expressed on certain T cell subsets and on subsets of macrophages and NK cells, but not on resting B cells. Ly6C is up regulated upon LPS stimulation of B cells.

Twenty-four hours following EIU induction, flow-cytometry revealed a robust inflammatory response in the LPS-injected eyes compared with controls, determined by increased levels of CD45⁺ cells out of all sampled cells (1.11±1.94% in control vs. 9.6±4.2% in EIU, p=0.002) and an increased fraction of D45⁺CD11b⁺ cells (63±10% in control vs. 95±3% out of all leukocytes in EIU, p<0.001; FIG. 4A, middle panel, FIG. 4B). The main effector cells 24 h post-EIU were CD45⁺CD11b⁺Ly6G⁺ cells, representing neutrophils, which were significantly increased after LPS injection (20±17% vs 51±13% out of sampled leukocytes, p=0.01, data not shown). 3K3A-APC pretreatment significantly reduced leukocyte percentage compared with untreated EIU eyes (4.1±3.6% vs. 9.6±4.2%, respectively. p=0.019; FIG. 4A, lower panel, FIG. 4B).

### Example 3

### The Effect of 3K3A-APC on Local Immune Environment and Activation of Resident Microglia

Microglia are the key immune effector cells of the central nervous system (CNS), including the brain, spinal cord and retina. They are the resident macrophage population of the CNS. Adequate microglial function is crucial for a healthy CNS as they regulate CNS development, maintenance of neuronal networks, and injury repair. Microglia are distinct from other tissue macrophages owing to their unique homeostatic phenotype and tight regulation by the CNS microenvironment. They are responsible for the elimination of microbes, dead cells, redundant synapses, protein aggregates, and other particulate and soluble antigens that may endanger the CNS. Furthermore, as the primary source of proinflammatory cytokines, microglia are pivotal mediators of neuroinflammation and can induce or modulate a broad spectrum of cellular responses. Alterations in microglia functionality are implicated in brain development and aging, as well as in neurodegeneration. Surveying or “resting” microglia are ramified and build a dense network spanning the CNS. Through highly motile long cellular processes they actively screen the microenvironment for disruptions in homeostasis. In response to infectious pathogens, injurious protein aggregates or tumor cells, microglia can initiate a neuroinflammatory response. Profound morphological and molecular changes accompany microglial activation. Injuries or inflammatory stimuli induce microglia to morph from a ramified to an amoeboid shape. Cell bodies enlarge while cell processes become shortened and cover more limited areas. Amoeboid morphology reflects a highly activated state associated with phagocytosis and proinflammatory function.

In surveying state, microglia express the markers Iba-1, CD68, CD11b, CD40, CD45, CD80, CD86, F4/80, TREM-2b, CXCR3 and CCR9.

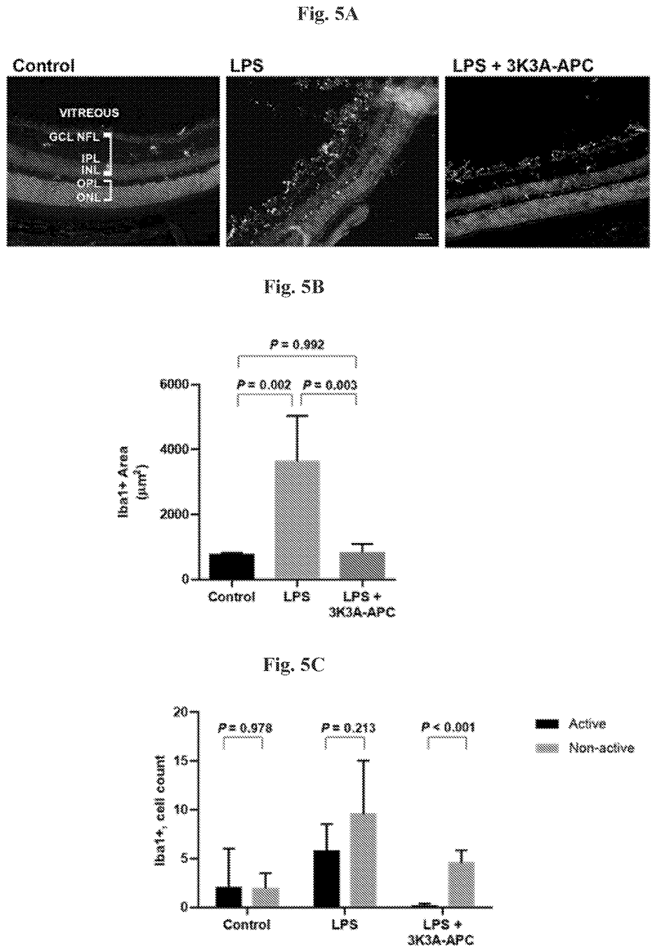

The microglia specific marker, ionized calcium-binding adapter molecule 1 (Iba1) was used for detecting microglia involvement in LPS-induced inflammation and response to 3K3A-APC treatment. The amount, morphology and anatomical location of microglial cells in the retina was assessed using confocal imaging of retinal cryosections. Representative images of control, LPS, and LPS+3K3A-APC treated eyes are presented in FIG. 5A. As expected, only a few microglial cells were detected in control eyes (Iba1 is stained green). Without inflammatory stimulation, microglia were located mainly in the inner retina (GCL, IPL) and had a ramified morphology, consistent with a non-inflammatory state. A dramatic increase in microglial cells was noted in the EIU eyes. Notably, the appearance of microglial cells at the outer layers of the retina presented an inflammatory amoeboid shape, indicating an activated phenotype. 3K3A-APC treatment restricted the increase in microglial amount and activation.

Quantitative analysis of microglia staining area revealed that LPS induced a statistically significant increase in Iba1 positive area, while 3K3A-APC treatment prevented this increase (FIG. 5B). Moreover, the total number of microglia cells at the outer retinal segments was counted, namely, microglia cells were assessed at the outer nuclear and outer plexiform layers, where microglia cells do not normally reside under physiologic conditions. In these areas, LPS increased total cell number compared to controls (15.25±3.38 vs. 3.94±3.06; p<0.001). Significantly, treatment with 3K3A-APC resulted in a lower number of microglia cells in outer retina compared with 3K3A-APC untreated eyes (LPS) (4.63±1.45 vs. 15.25±3.38; p<0.001).

Next, microglia activation state was determined at the outer retinal segments by their morphology as activated (amoeboid) versus non-activated (ramified). FIG. 5C summarizes the quantitative analysis of the distribution between active and non-active microglial cells. In control and EIU eyes, there was no difference between the numbers of activated and non-activated cells. However, eyes treated with 3K3A-APC demonstrated a significantly lower number of activated microglial cells compared with the ramified microglia subpopulation (p<0.001). These results indicate that 3K3A-APC not only reduces microglial cell accumulation in the eye but also inhibits an activated microglial state.

### Example 4

The Effects of 3K3A-APC on NLRP3 Activation and IL1§ Expression after EIU Induction

The NLRP3 inflammasome is a crucial component of the innate immune response, whose activation ultimately results in the release of the pro-inflammatory cytokines interleukin (IL)-1(3 and IL-18. APC has been shown to inhibit murine NLRP3 inflammasome in injury models such as cardiac ischemia-reperfusion injury and ischemic white matter stroke. In the present study, the ability of 3K3A-APC treatment to decrease NLRP3 activation in the retina was evaluated.

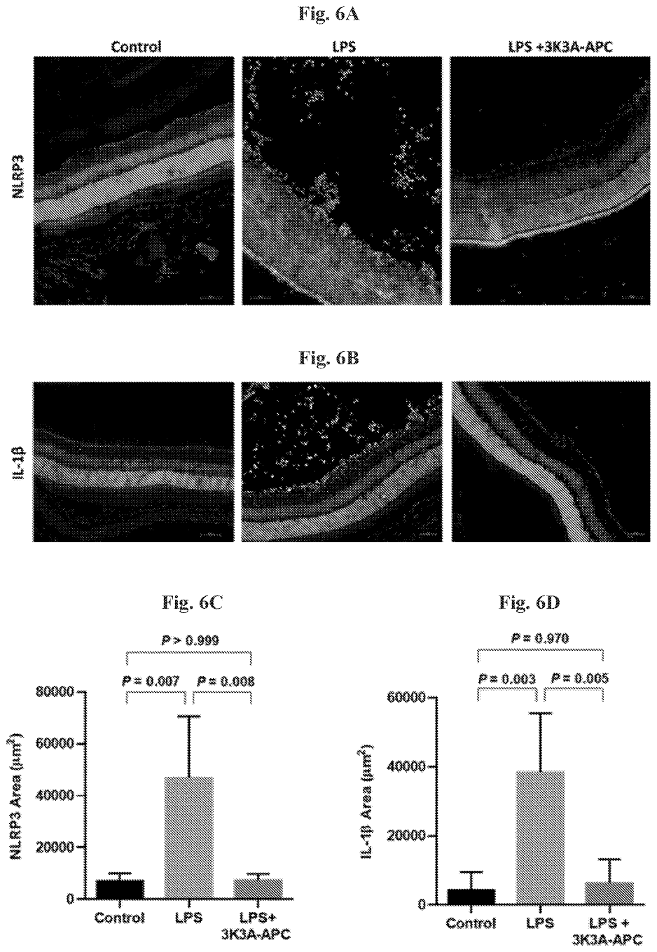

Twenty-four hours post-EIU induction, retinal cryosections were prepared and stained for NLRP3 and IL-1β. Representative images shown in FIGS. 6A-6B demonstrated minimal staining for NLRP3 and IL-1β, respectively in the control eyes. Strong NLRP3 staining in the photoreceptor, outer plexiform and ganglion cell layers were noticed following LPS injection, but these were all attenuated when 3K3A-APC injection preceded LPS injection, indicating that 3K3A-APC pre-treatment prevented the LPS-induced increase in NLRP3 expression. Abundant cells positive for IL1β staining in the vitreous and inner retinal layers are seen in LPS treated eyes, while eyes pre-treated with 3K3A-APC demonstrate weak staining in the inner retinal layers. Compared with control eyes, an increase in IL-1β positive cells was observed after EIU with marked expression in the vitreous cavity. IL-1β secretion was prevented in eyes treated with 3K3A-APC prior to LPS injection, concurrent with NLRP3 activity reduction.

Quantitative analysis of NLRP3 and IL-1β (FIGS. 6C and 6D, respectively), confirmed the statistical significance of NLRP3/IL-1β elevation following EIU induction that was almost entirely prevented by 3K3A-APC treatment.

### Example 5

### The Effect of Intravenously Administered 3K3A-APC on LPS-Induced Ocular Inflammation

To evaluate the potential of intravenously injected recombinant murine 3K3A-APC to cross the blood-retina barrier and induce ocular protective effects, the lipopolysaccharide (LPS)-induced ocular inflammation (EIU) murine model was used in order to assess 3K3A-APC protective activities in the retina following its injection into the mice's tail.

The concentration of 3K3A-APC (0.5 mg/kg) and frequency of the injections were determined based on previous publications (Wang et al., Front Neurosci., 16:841916, 2022; Huuskonen et al., J Exp Med., 219(1):e20211372, 2022; Lazic et al., J Exp Med., 216(2):279-293, 2019; Lyden et al., Curr Pharm Des., 19:7479-7485, 2013; Lyden et al., Ann Neurol., 85(1):125-136, 2019).

The ability of intravenously administration via tail vein of 3K3A-APC to inhibit inflammatory cells extravasation into the retina parenchyma and increase their retention in blood vessels was studied using the following study groups, each consisting of 3 mice:
(i) naïve control group: venous injection of saline in the same regimen (concentration and frequency) as 3K3A-APC. No intravitreal injection of LPS;
(ii) ocular inflammation group: intravitreal injection of LPS followed by venous injection of saline at two time points: 10 minutes and 4 hours after administration of LPS; and
(iii) treatment group: LPS intravitreal injection followed by venous injection of 3K3A-APC (0.5 mg/ml) at two time points: 10 minutes and 4 hours after administration of LPS.

Twenty-four hours post-treatment, in-vivo injection of fluorescein isothiocyanate (FITC)-dextran was performed to fluorescently mark retinal blood vessels. Immediately thereafter, retinas were isolated and prepared as flatmounts. Additional immunostaining for CD11b was performed to allow the detection of inflammatory cells inside blood vessels and in the retinal parenchyma. FIG. 7 shows representative color images of CD11b (red) and blood vessels (green) staining in flatmounts of control, LPS and LPS+3K3A-APC treated eyes. As seen, intravenously injected 3K3A-APC dramatically reduced immune cell numbers in the retina and inhibited their extravasation from blood vessels.

These results clearly indicate that intravenous administration of 3K3A-APC can induce anti-inflammatory activities within the retina and suggest that 3K3A-APC may cross the blood retina barrier.

### Example 6

### The Effect of Intravenously Administered 3K3A-APC on Laser-Induced Choroidal Neovascularization (CNV)

The feasibility of intravenous administration of 3K3A-APC to inhibit the growth and penetration of pathological blood vessels from the choroid into the sensory retina and reduce the leukocyte number and extravasation into the retina was assessed. The study included 8-weeks-old male C57BL/6J mice weighing 19 to 25 grams.

The following study groups, altogether employing 13 mice, were used:
(i) control group: venous injection with 3K3A-APC. No Laser treatment
(ii) CNV group: laser induced CNV followed by venous saline injection 1 hour and 3 days laser application; and
(iii) treatment group: laser-induced CNV followed by venous injection of 3K3A-APC (0.2 mg/kg) 1 hour and 3 days after laser application.

Choroidal neovascularization was induced by laser as described in Material and Methods. Ten days post-laser application, CNV was stained green using FITC-dextran perfusion, and flat retinal pigment epithelium (RPE)-choroid specimens were isolated and stained red with anti-CD11b antibodies. The retinal RPE and choroid are complex tissues that provide crucial support to the retina. The RPE has multiple functions such as absorption of light. The choroid is a heterogeneous connective tissue that supports both the RPE and the outer retina.

The images of CD11b (red) and blood vessels (green) staining in FIG. 8A, show that 3K3A-APC reduced immune cell numbers and inhibited CNV growth toward the RPE surface.

CNV depth (μm), and CD11b positive cell number were quantified, and the results are presented in the bar graphs shown in FIGS. 8B-8C.

The results suggest that the intravenous administration via the tail vein of 3K3A-APC induced a statistically significant reduction in CNV development and penetration from the choroid to the RPE surface. Moreover, systemic administration of 3K3A-APC reduced the accumulation of inflammatory cells accompanying CNV development.

The results suggest that systemic administration of 3K3A-APC can potentially treat ocular pathologies associated with inflammation and/or CNV development.

### Example 7

### The Effect of 3K3A-APC on NLRP3 Levels at a CNV Lesion

The impact of 3K3A-APC treatment on NLRP3 levels at the CNV sites was assessed. The study included 8-weeks-old male C57BL/6J mice weighing 19 to 25 grams. Choroidal neovascularization was induced by laser, and 3K3A-APC was ITV administered as described in Material and Methods.

One hour after CNV induction, either 3K3A-APC or saline was intravitreally injected, and four days later, retinal cryosections were taken and stained for NLR family pyrin domain containing 3 (NLRP3) as described in Material and Methods. Representative images of retinal cryosections immunostained with NLRP3 antibodies (green) are shown in FIG. 9A. As seen, in the left image, minimal staining of NLRP3 was detected in control eyes that were not subjected to laser. NLRP3 staining was mostly restricted to the outer part of the retina, with very little observed in the outer and inner plexiform layers (OPL and IPL, respectively). This staining pattern reflects the constitutive expression of NLRP3 inflammasome in various cell types, including the RPE, retinal microglia, Müller cells, and astrocytes. In eyes subjected to laser and treated with saline, prominent staining for NLRP3 was observed in the CNV area, extending to the RPE and throughout all retinal neurosensory layers, including the OPL and IPL. Notably, NLRP3 expression was not only limited to the CNV site itself but also extended to the margins of the CNV lesion (middle and right images in the upper panel in FIG. 9A). In contrast, eyes subjected to laser and treated with 3K3A-APC showed a significant attenuation of NLRP3 staining, with minimal staining observed at the CNV site (marked with an asterisk), resembling the staining pattern of control eyes without CNV (lower images in FIG. 9A). Quantitative analysis of NLRP3 areas confirmed the statistical significance of these findings, as illustrated in the box-and-whiskers plot shown in FIG. 9B. The increase in NLRP3 levels observed following CNV induction was effectively prevented by 3K3A-APC treatment (median 0.32 [IQR 0.27-0.51]μm² vs. 0.05 [IQR 0.04-0.07]μm², respectively, p=0.020), reaching levels comparable to those observed in control eyes (median 0.05 [IQR 0.04-0.15]μm², p >0.999). The interquartile range (IQR) is the width of the box namely, IQR=Quartile 3−Quartile 1.

### Example 8

### The Effect of 3K3A-APC on IL-1β Levels at a CNV Lesion

The NLRP3 inflammasome activation ultimately results in the release of the pro-inflammatory cytokines interleukin (IL)-1β and IL-18. To evaluate the ability of 3K3A-APC to inhibit inflammasome activation, IL-1β levels were measured at CNV sites. Laser-induced CNV and intravitreal administration of 3K3A-APC were conducted as described in Material and Methods.

One hour after CNV induction, either 3K3A-APC or saline was intravitreally injected, and four days later, retinal cryosections were prepared and stained for IL-1β as described in Materials and Methods. Representative images of retinal cryosections immunostained with IL-1β antibodies (green) are shown in FIG. 10A. As seen in the left image, in control eyes, minimal IL-1β staining was observed. However, in eyes with CNV treated with saline a pronounced elevation in IL-1β staining was observed at the CNV site (marked with an asterisk), at the RPE and all neurosensory retinal layers, including the OPL and IPL (middle and right images in the upper panel in FIG. 10A). In contrast, eyes with CNV lesion treated with 3K3A-APC showed almost no IL-1β staining in the CNV area or throughout the retina layers (two lower images in FIG. 10A), indicating a significant reduction in IL-1β levels with 3K3A-APC treatment. Quantitative analysis presented as box-and-whisker plot in FIG. 10B, showed a statistically significant elevation of IL-1β following CNV induction that was dramatically reduced by 3K3A-APC treatment (median 0.35 [IQR 0.27-0.78]μm² vs. 0.08 [IQR 0.02-0.13]μm², respectively, p=0.022)), reaching levels comparable to those observed in control eyes (median 0.04 [IQR 0.01-0.11]μm², p >0.999).

### Example 9

### The Effect of 3K3A-APC on Microglia Recruitment and Activation at a CNV Lesion

The presence of microglia cells at the sites of a CNV lesion, and their activation state were assessed based on evaluating their specific marker Iba1 (ionized calcium-binding adaptor molecule 1) and their morphology characterized as either activated (amoeboid shape) or non-activated (ramified shape). Iba1 is a reliable marker for microglial activation. Laser-induced CNV, intravitreal administration of 3K3A-APC and immunostaining of Iba1⁺ cells were conducted as described in Material and Methods.

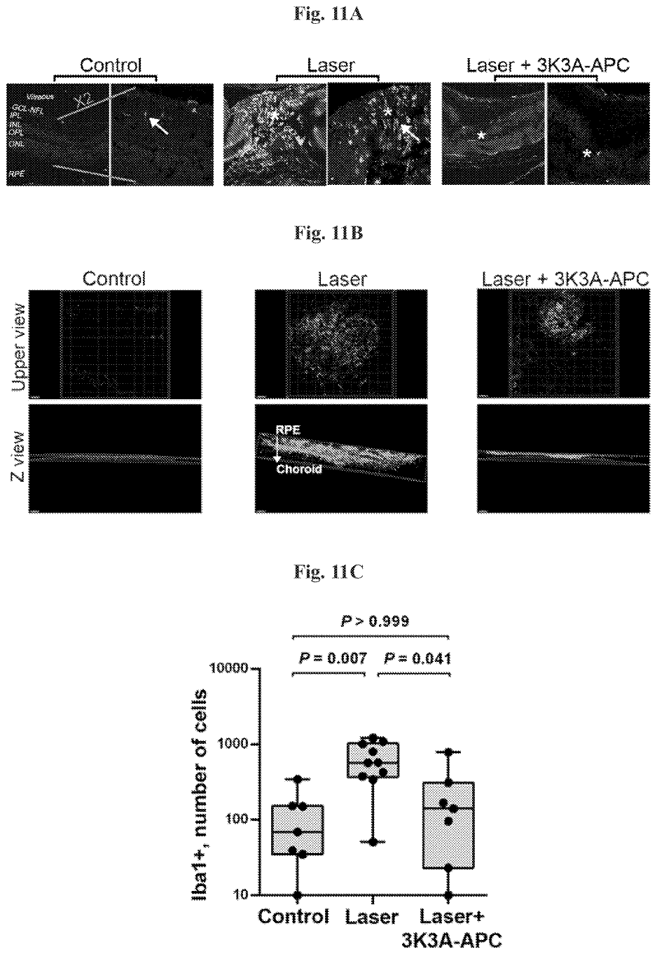

One hour after CNV induction, either 3K3A-APC or saline was injected intravitreally. Four days later, retinal cryosections were prepared and stained for Iba1. Representative images of retinal cryosections immunostained with Iba1 antibodies (green) and DAPI (blue) as a nuclei marker, are shown in FIG. 11A. As seen in left couple of images, control eyes, without any intervention, showed scant and ramified (non-active, example marked by an arrow) Iba1⁺ cells, mainly in the inner retinal layers. In the untreated eyes with CNV lesion, a significant increase in microglial cells was observed in the CNV area and the surrounding retina (FIG. 11A, 2 image in the middle). Furthermore, these cells underwent a morphological change towards an amoeboid shape, indicating their activation (example marked by an arrow). However, in eyes treated with 3K3A-APC after laser induction of CNV, both a reduction in the number of microglial cells and inhibition of their activation, indicated by ramified morphology, were detected (FIG. 11A, right couple of images).

The presence of Iba1⁺ cells and their response to 3K3A-APC was further assessed in RPE-choroid flatmounts. Intravitreal injections of either 3K3A-APC or saline were administered one hour after CNV induction. One week later, RPE-choroid specimens were prepared as flatmounts and stained with anti-Iba1 antibodies as described in Materials and Methods. The specimens were positioned with the RPE layer facing upwards and the choroid resting on the slide and were scanned using confocal microscopy from the RPE into the choroid. FIG. 11B shows representative upper-view (upper panels) and depth Z-plane (lower panels) color images of the RPE-choroid flatmounts. As seen, minimal staining of Iba1⁺ cells was detected in control eyes not subjected to laser applications (left images). However, in eyes exposed to laser and treated with saline, deeper and more extensive accumulation of Iba1⁺ cells was observed from the RPE surface throughout the depth of the choroid (middle images). Treatment with 3K3A-APC significantly reduced the accumulation of Iba1⁺ cells in CNV sites (right images). Notably, similar to the observation in the cryosection, the Iba1⁺ cells were located at the edge of the RPE rather than deeper in the choroid.

Quantitative measurements of Iba1⁺ cell counts in the entire RPE-choroid specimen, presented as box-and-whisker plot in FIG. 11C, showed a significant reduction in the median count of Iba1⁺ cells in the 3K3A-APC-treated eyes compared to the untreated eyes (141 [IQR 23-311] vs. 569 [IQR 367-1032] cells, p=0.041, respectively). These findings indicate that 3K3A-APC treatment restricted the accumulation and activation of microglia cells within the site of the CNV lesion.

### Example 10

### The Effect 3K3A-APC Treatment on Myeloid Cells Accumulation and CNV Growth

The effect of 3K3A-APC treatment on myeloid cells accumulation when administered after established CNV-related inflammation is already present, was assessed. As robust inflammation was observed on days 4-7 after CNV induction (FIGS. 9-11), CNV induced in eyes which were not treated with the APC variant), a treatment regimen of two consecutive intravitreal injections of 1 μg/μl/eye 3K3A-APC was used, wherein 3K3A-APC was administered on days four and seven post-CNV induction. The concentration of 3K3A-APC was chosen based on previously performed studies. Seven days after the last treatment (on day 13), an in-vivo injection of fluorescein isothiocyanate (FITC)-dextran was performed to mark retinal blood vessels fluorescently. Immediately thereafter, RPE-choroid specimens were prepared as flatmounts, stained with the myeloid marker CD11b, and scanned using confocal microscopy. The timeline of the experiment is schematically shown in FIG. 12A. Representative images of stained CD11b⁺ cells and perfused CNV vasculature of RPE-choroid flatmounts are shown in FIG. 12B. Upper view and depth Z-plane are presented (upper panel and lower panel, respectively, in FIG. 12B). In eyes not subjected to any intervention (“Control”; 2 stacked images on the left), neither presence of CD11b⁺ cells nor CNV's vascular component was found. Eyes subjected to CNV induction with saline treatment (“Laser”; 2 stacked images in the middle) showed a significant presence of CD11b⁺ cells throughout the CNV depth, which reduced significantly upon 3K3A-APC treatment (“Laser+3K3A-APC”; two stacked images on the right).

Quantification of CD11b⁺ cells in the RPE-choroid specimens is depicted as box-and-whiskers plot shown in FIG. 12C. As expected, almost no CD11b⁺ staining was observed in eyes not subjected to CNV induction (median of 7 [IQR 2-10] cells). Eyes with laser-induced CNV and vehicle treatment showed markedly elevated CD11b⁺ cell numbers in the tissue (median of 215 [IQR 98-370] cells). 3K3A-APC treatment dramatically reduced CD11b⁺ cell count (median 8 [IQR 3-58] cells, p=0.017), similar to eyes not subjected to laser application (p >0.999). The total volume of CNV was significantly regressed by 3K3A-APC treatment. The median volume of CNV was 7342 μm³ (IQR 1054-27904) in saline-treated eyes, whereas it was 0 μm³ (IQR 0-513) in 3K3A-APC-treated eyes (p=0.008). Similarly, the median depth of CNV was 20 μm (IQR 7.5-28) in saline-treated eyes, compared to 0 μm (IQR 0-5) in 3K3A-APC-treated eyes (p=0.026), as shown in FIG. 12D.

These results corroborate earlier findings by the present inventors that 3K3A-APC has the ability to suppress CNV growth and penetration from the choroid into the retina and demonstrate that treatment with 3K3A-APC reduces the involvement of inflammatory cells in the neovascular process.

### Example 11

The Effect of 3K3A-APC Treatment on Leakage from CNV and VEGF Levels

In clinical scenarios, such as nAMD, increased permeability of the neovessels can result in excessive fluid accumulation and hemorrhage into the surrounding tissues, ultimately leading to vision loss. These scenarios were simulated, the protective role of 3K3A-APC against leaking CNV lesions was evaluated. Furthermore, it was determined whether leakage assessed by fluorescein angiography (FA) can be a surrogate marker for in-vivo evaluation of 3K3A-APC treatment efficacy.

For these purposes, CNV growth through laser photocoagulation was first induced in mice, and fluorescein sodium was IP injected as described in Materials and Methods. Pre-treatment FA was conducted on day 4 to confirm the presence of leakage from CNV, as can be detected in FA performed in humans. Mice with confirmed leakage were then divided into two groups: 3K3A-APC or saline treatment. The chosen treatment regimen was based on the consecutive injections applied in pre-clinical trials of 3K3A-APC (Huuskonen M. T. et al., J. Exp. Med., 219, 2022; Lazic D. et al., J. Exp. Med., 216: 279-293, 2019) and the common practice with anti-VEGF injections. Therefore, the treatment regimen applied included two consecutive intravitreal injections of 1 μg/μl/eye 3K3A-APC, administered on day four and day seven post-CNV induction. Eleven days post-laser, additional FA was performed to assess the efficacy of 3K3A-APC treatment on CNV leakage. The experiment timeline is depicted in FIG. 12A. For dynamic FA assessment, sequential real-time images of the same eye were captured during the early phase (during the first minute from fluorescein injection) and late phase (every minute between 2 to 5 minutes following fluorescein injection). The FA images obtained were analyzed by masked retina specialists that evaluate leakage from CNV. The results of the study are presented in FIGS. 13A-13B.

FIG. 13A displays representative dynamic FA images of 3K3A-APC and saline-treated eyes. On day 4, before treatment, dynamic FA imaging showed hyperfluorescence at lesion sites in both groups (i.e., before 3K3A-APC or saline treatment) on early images, which intensified with the blurring of margins on late images, indicating active, leaking CNV lesions (FIG. 13A, left cluster of 4 images). On day 11, imaging of saline-treated eyes showed active leakage from CNV (FIG. 13A, right 2 upper images in the cluster of 4 images). In contrast, 3K3A-APC-treated eyes showed scant early hyperfluorescence that was stable without intensified hyperfluorescence and no blurring of margins, indicating non-active (or scarred) lesions (FIG. 13A, right 2 lower images in the cluster of 4 images).

A quantitative assessment comparing the percentage of leaking lesions in 3K3A-APC or saline-treated eyes, performed on day 11, is presented in FIG. 13B. Pathologically significant leakage was present in 11 out of 18 CNV lesions in saline-injected mice, but only in 1 out of 14 CNV lesions in 3K3A-APC-treated mice (61% vs. 7%). The analysis showed that the odds of forming non-leaking lesions increase by 20.43 (95% Cl 2.19-190.50, p=0.008) if 3K3A-APC is injected (Table 1).

TABLE 1
Effect of 3k3A-APC on the status of laser lesions estimated by a generalized
estimating equation

| Dependent | Independent | | | | Odds | 95% CI for Odds |
|---|---|---|---|---|---|---|
| variables | variables | B | SE of B | P value | Ratio | Ratio |
| Non-leaking lesion | Intercept | −0.45 | 0.55 | 0.412 | 0.64 | 0.22 |
| | 3K3A-APC | 3.02 | 1.14 | 0.008 | 20.43 | 2.19 |
| | treatment | | | | | |

In the generalized estimating equation, the lesion status (“leaking” or “non-leaking”) is a binary outcome and 3K3A-APC treatment (“no” or “yes”) is a between-subjects factor. B: unstandardized coefficient; SE: standard error; Cl: confidence interval.

Moreover, a statistical analysis of the relationship between the leakage status of each specimen and its vascular endothelial growth factor (VEGF) levels (quantified by VEGF immunostaining in retinal flatmounts) showed that the mean VEGF volume in non-leaking lesions was 4607 μm³ vs. 131529 μm³ in leaking lesions (p=0.009, Table 2). These findings indicate a strong correlation between leakage and elevated VEGF levels.

TABLE 2
Relationship between the lesion status (“leaking”
or “non-leaking”) and VEGF volume, estimated
by a linear mixed model with random intercept

| Dependent | Independent | | | |
|---|---|---|---|---|
| variables | variables | B | SE of B | P value |
| VEGF volume | Intercept | 4607 | 26781 | 0.865 |
| | Leaking lesion | 131529 | 47116 | 0.009 |

### Example 12

### The Neuroprotective Effect of 3K3A-APC

The optic nerve crash (ONC) mice model is a well-validated model of optic neuropathy. It is a valuable preclinical model for glaucoma and ischemic optic neuropathy (ION), useful in studying neuronal survival and regeneration. Optic nerve crush induces significant retinal ganglion cells (RGCs) death with little variability and severs all axons, thereby assuring that any fibers found past the injury site are regenerating rather than spared. Optic nerve crush has been associated with the activation of retinal microglia and neuroinflammation. The ONC mice model was used to focus on 3K3A-APCs' ability to inhibit neurodegenerative key pathways and demonstrate that 3K3A-APC inhibits RGCs loss.

Optic nerve crash was induced in C57BL/6 mice by applying forceps 2.5 to 3.0 mm posterior to the globe for 7 seconds. This procedure was repeated three times.

Murine recombinant 3K3A-APC (1 μg/μl/eye) or saline was injected intravitreally one hour after ONC and a week later. Fourteen days after ONC induction, the eyes were enucleated. Retinal cryosection and retinal flatmount were prepared as escribed in Materials and Methods and used for immunolabeling assays. RGC loss was evaluated using RNA binding protein (RBPMS), a specific marker for RGC. Microglia cell number and localization were assessed using Iba1 immunostaining.

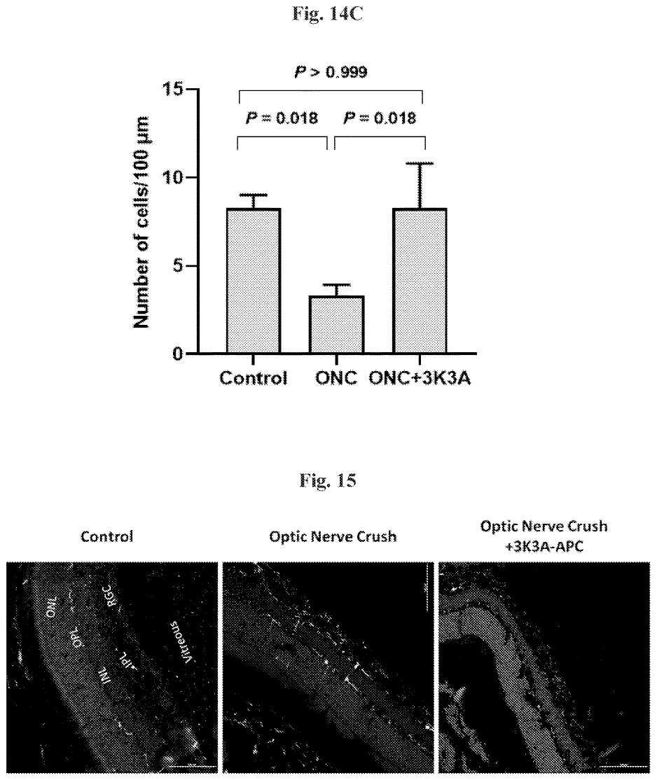

Representative retinal flatmount images taken 14 days post-ONC, with or without 3K3A-APC treatment, are shown in FIG. 14A. The left image demonstrates the massive RGCs loss induced by ONC. The right image shows that 3K3A-APC treatment inhibited the RGCs loss. The viable RGCs were stained pale blue using RBPMS antibody.

Representative retinal cryosections images taken 14 post-ONC, with or without 3K3A-APC treatment, are shown in FIG. 14B. As seen, the massive RGCs loss induced by ONC was inhibited by 3K3A-APC treatment.

The total number of RGC cells in cryosections was quantified and the results are presented in the bar graph shown in FIG. 14C. Bars demonstrate a statistically significant decrease in RGCs number induced by ONC, which was prevented by 3K3A-APC treatment. RGCs positive area was calculated using values of 4 microscopic fields (2 fields×2 slides), which were averaged and used as raw data for further analysis.

Microglial cells in the retinal cryosection (taken 14 days post ONC) were stained for Iba1, a specific marker for microglia. The results are shown in FIG. 15. Control image (left image) demonstrated scarce microglia (stained green) mainly in the inner retina, having a ramified morphology indicating a non-inflammatory state. Blue staining is DAPI staining of the nuclei. ONC increased the total amount of Iba1 positive cells and their translocation to the outer retinal layers, and induced a shape change as seen in the middle image. With 3K3A-APC treatment (right image), a reduction in Iba1 positive staining was noted and cells resumed a more ramified morphology.

1. A method for treating an ocular disease, disorder of condition selected from the group consisting of:
(i) inflammation in the eye;
(ii) an ocular pathology associated with activation of retinal NLRP3 inflammasome;
(iii) an ocular pathology associated with translocation and activation of retinal microglia cells;
(iv) a disease-associated macular edema; and
(v) ischemic retinopathy,
comprising administering a therapeutically effective amount of a variant of activated protein C (APC) or a functional partial sequence thereof to a subject in need thereof, thereby treating said disease, disorder of condition in the.

2. The method of claim 1, wherein the inflammation is associated with an ocular or non-ocular disease, disorder or condition, caused directly by inflammation, features development of inflammation as a secondary stage or a complication thereof, or features inflammation as a synchronous or asynchronous sequela thereof.

3-5. (canceled)

6. The method of claim 1, wherein:
the ocular disease is at least one of: anterior, intermediate and posterior uveitis, panuveitis, endogenous and exogenous endophthalmitis, inflammatory diseases of the optic nerve selected from the group consisting of optic neuritis, papilledema, anterior and ischemic optic neuropath (AION), age-related macular degeneration (AMD), particularly, nAMD (wet AMD), pathologic (high) myopia, and pseudoxanthoma elasticum with angioid streaks, Behçet's disease, retinitis pigmentosa, glaucoma, best Vitelliform macular degeneration (BVMD), Stargard's disease, choroiditis, episcleritis, scleritis, thyroid ophthalmopathy and retinopathy; and
the ocular disorder or condition is at least one of: choroidal neovascularization (CNV), retinal leakage, blood vessel occlusion, oxidative damage, damage to the optic nerve and the death of associated retinal ganglion cells (RGCs) by elevated intraocular pressure (IOP), chronic inflammation, an autoimmune condition sarcoidosis systemic lupus erythematosus (SLE), temporal arteritis, giant cell arteritis (GCA)), Lyme disease, viral infection, cat scratch fever (Bartonella), syphilis, bacterial infection, Herpes virus, exogenous or endogenous endophthalmitis, an accidental, occasional incidence in which inflammation develops following a traumatic injury of the retina, perforating and blunt trauma, complications during or post ophthalmic medical procedure, or drugs side effects.

7. The method of claim 6, wherein the ocular disease or the ocular pathology is selected from the group consisting of a retinal disease disorder or condition, is anterior, intermediate and/or posterior uveitis, CNV and AMD.

8-11. (canceled)

12. A method for ocular cytoprotection, comprising administering to a subject in need thereof a therapeutically effective amount of a variant of activated protein C (APC) or a functional partial sequence, thereby cytoprotecting the eye of the subject.

13. The method of claim 12, wherein cytoprotection is neuroprotection.

14. The method of claim 12, for treating neurodegenerative disorders of the retina, optic nerve injury, death of retinal ganglion cells (RGCs), exposure to toxins and genetic mutations.

15. (canceled)

16. The method of claim 1, wherein the disease-associated macular edema is at least one of diabetic macular edema, retinal vein occlusion (RVO), retinal artery macroaneurysms (RAM), postoperative cystoid macular edema, radiation retinopathy, non-arteritic anterior ischemic optic neuropathy (NAION), uveitis associated macular edema, age-related macular degeneration (AMD), hypertensive retinopathy (stage IV), edema after panretinal photocoagulation (PRP), drug-induced macular edema, choroidal tumors, retinitis pigmentosa, hereditary/genetic disorders, macular edema associated with an inflammatory disease or disorder of the immune system selected from cytomegalovirus infection, retinal necrosis, sarcoidosis, Behçet's syndrome, toxoplasmosis, Eales' disease or Vogt-Koyanagi-Harada syndrome, or injuries.

17. (canceled)

18. The method of claim 1, wherein the ischemic retinopathy is at least one of: central retinal artery occlusion (CRAO), branch retinal artery occlusion (BRAO), central retinal vein occlusion (CRVO), branch retinal vein occlusion (BRVO), proliferative and non-proliferative diabetic retinopathy (DR), retinal vasculitis infections or inflammation, or retinopathy of prematurity (ROP).

19. The method of claim 1, wherein the APC variant or a functional partial sequence thereof are administered systemically to the subject.

20. The method of claim 19, wherein the APC variant or a functional partial sequence thereof are injected intravenously.

21. The method of claim 1, wherein the APC variant or a functional partial sequence thereof is administered directly to the eye of the subject.

22. The method of claim 21, wherein the APC variant or a functional partial sequence thereof is intravitreally injected and/or topically applied to the eye of the subject.

23. The method of claim 1, wherein the effective amount is from about 50 μg/kg body weight to about 700 μg/kg body weight, at least one of 120, 240, 360, or 540 jug/kg body weight of APC mutant or a functional partial sequence thereof.

24-25. (canceled)

26. The method of claim 1, wherein the amino acid sequence of the APC variant is at least 70%, at least 80%, at least 85%, at least 90%, at least 95%, or at least 98%, identical to the amino acid sequence of wild type APC, and it maintains at least part of the wild type APC activity.

27. The method of claim 1, wherein the APC variant is one or more of: 3K3A-APC, RR229/230AA-APC, 5A-APC, APC-2Cys, K193E-APC, E149A-APC, a wild type APC in which residue 158 (Asp) is substituted with a non-acidic amino acid, or residue 154 (His) is substituted with an amino acid residue selected from the group consisting of Lys, Arg or Leu.

28. The method of claim 27, wherein the APC variant is 3K3A-APC.

29. The method of claim 1, further comprising administration of one or more active agents selected from the group consisting of an anti-angiogenesis, anti-inflammatory, anti-bacterial, immunosuppressive, anti-platelet derived growth factor (PDGF), anti-fungal, anti-viral agent and adeno-associated virus (AAV)-based gene therapy.

30. The method of claim 29, wherein the active agent is one or more of: an anti-VEGF, steroid, angiopoietin-Tie2 signaling pathway drug, bi-specific antibody targeting VEGF and Ang2, and NLRP3/IL1β inhibitor.
